(19) United States
(12) Patent Application Publication
(10) Pub. No.: US 2006/0123518 A1
(43) Pub. Date: Jun. 8, 2006

(54) Methods for culturing cereal endosperm

(75) Inventors: Odd-Arne Olsen, Johnston, IA (US); Hena Guo, Johnston, IA (US); Darren B. Gruis, Baxter, IA (US)

Correspondence Address: ALSTON & BIRD LLP;PIONEER HI-BRED INTERNATIONAL, INC., BANK OF AMERICA PLAZA, 101 SOUTH TRYON STREET, SUITE 4000, CHARLOTTE, NC, 28280-4000, US

(73) Assignee: Pioneer Hi-Bred International, Inc., Johnston, IA (US)

(21) Appl. No.: 11/289,937

(22) Filed: Nov. 30, 2005

Related U.S. Application Data
(60) Provisional application No. 60/632,155, filed on Dec. 1, 2004.

(51) Int. Cl. A01H 5/00; C12N 5/04

(52) U.S. Cl. 800/320.1; 435/412

(57) ABSTRACT

The present invention provides in vitro methods for culturing cereal endosperm that allow for the development of the endosperm in a manner that is analogous to endosperm development in planta. The methods involve isolating fertilized embryo sacs and exposing the embryo sac or portion thereof to a plant culture medium for an extended period of time so as to promote the growth and development of the endosperm therein or the growth and development of both the endosperm and embryo therein. The invention further provides methods for introducing polypeptides and polynucleotides into the cultured fertilized embryo sacs or portions thereof.

### CROSS-REFERENCE TO RELATED APPLICATIONS

This application claims the benefit of U.S. Provisional Application No. 60/632,155, filed Dec. 1, 2004, which is hereby incorporated herein in its entirety by reference.

### FIELD OF THE INVENTION

This invention relates to the field of biotechnology, particularly to in vitro methods for culturing plant tissues.

### BACKGROUND OF THE INVENTION

Among the world's most important crops are the cereals, rice, wheat, and maize. The endosperm is by far the predominant tissue in mature cereal seeds and thus, a thorough understanding of endosperm development is of great interest and importance in agriculture (Kowles and Phillips (1988) Int. Rev. Cytol. 112:97-136; Olsen et al. (1992) Seed Sci. Res. 2:117-131; Lopes and Larkins (1993) Plant Cell 5:1383-1399; Clore et al. (1996) Plant Cell 8:2003-20140; Olsen (2001) Annu. Rev. Plant Physiol. Plant Mol. Biol. 52: 233-267; Olsen (2004) Plant Cell 16: S214-S227).

A significant obstacle to studying endosperm development is the location of the endosperm within the plant and relative inaccessibility of the endosperm to experimental manipulation, particularly the early developmental stages that occur immediately after fertilization and several days thereafter (Goldberg et al. (1994) Science 266:605-614). In vitro culture systems may provide a way to overcome this significant obstacle and a variety of approaches have been reported including cultures that were initiated with embryo sacs that were isolated by a method involving the use of digestive enzymes (Wagner et al. (1989) Plant Sci. 59:127-132). Others have reported the initiation of in vitro cultures from mechanically isolated embryo sacs that also included surrounding maternal nucellus cells (Campenot et al. (1992) Am. J. Bot. 79:1368-1373. Leduc et al. ((1995) Sex. Plant Reprod. 8:313-317), and have indicated that the structural integrity of isolated embryo sacs is important for long-term viability and further that adjacent nucellus cells may be essential for stimulating development. In one recent approach, ovule sections containing intact embryo sacs were isolated by the mechanical sectioning of maize ovules with a Vibratome (Laurie et al. (1999) In Vitro Cell Dev. Biol.—Plant 35:320-325). When cultured on Murashige and Skoog media, the embryos germinated with and without the endosperm and were capable of producing plants. Laurie et al. ((1999) In Vitro Cell Dev. Biol.—Plant 35:320-325) also indicated that is it probable that the maternal tissues that surround the embryo sacs in the Vibratome-produced sections provide the zygote with essential factors for development in vitro. Furthermore, the Laurie et al. ((1999) In Vitro Cell Dev. Biol.—Plant 35:320-325) reported that the presence of the ovary wall and nucellus minimize possible damaging movement during required mechanical manipulations. More recently, these Vibratome-produced maize ovule sections have been reported to be used as a target tissue in methods for the transformation and regeneration of transformed maize plants (U.S. Pat. No. 6,300,543).

While the in vitro culture systems discussed above can be used to study the early stages of embryo and endosperm development, these systems do not allow scientists to study endosperm and embryo development in the absence of any significant influence from maternal tissues, particularly the ovary wall and/or nucellus. One approach to eliminate maternal influences on embryo development is through in vitro fertilization of isolated single egg and sperm cells. Kranz and Lorz ((1993) Plant Cell 5:739-746) reported the regeneration of maize plants following the in vitro fertilization of isolated single egg and sperm cells by electrofusion and subsequent culture of the fusion products on a medium comprising maize feeder cell suspensions to support the growth of the developing embryos. More recently, Kranz et al. ((1998) Plant Cell 10:511-524) reported the electrofusion of isolated maize sperm with central cells and the subsequent development of endosperm on a medium comprising maize feeder cell suspensions. However, such a system has not been demonstrated to be useful for studying the long-term development of the endosperm, particularly the differentiation of the aleurone layer and starchy endosperm cells. Furthermore, embryo and endosperm development in the in vitro culture systems of Kranz and Lorz ((1993) Plant Cell 5:739-746) and Kranz et al. ((1998) Plant Cell 10:511-524) may be influenced by the presence of the feeder suspension cells and the effects of multiple genotypes. Each of these in vitro culture systems involve the use of culture media containing feeder cells that were derived from a different maize genotype than the genotype of the maize plants from which the sperm, egg, and central cells were isolated.

Endosperm suspension cultures have been previously reported. Felker ((1987) Am. J. of Bot. 74:1912-19200) describes the in vitro endosperm suspension culture system that was first described by Shannon ((1982) “Maize endosperm cultures”, in Sheridan, W. F., ed., Maize for Biological Research, Plant Molecular Biology Association, Charlottesville, Va., pp 397-400). According to Felker, these cultures have been used as a model system to study biochemical events in developing seeds. Cultures derived from endosperm 10 days after pollination have been maintained on agar medium or in liquid suspension for several years. Unlike many plant cell cultures consisting of undifferentiated cells, maize endosperm cultures maintain some of their endosperm characteristics, including an ability to accumulate starch (Chu and Shannon (1975) Crop Sci. 15:814-819), zeins (Shimamoto et al. (1983) Plant Physiol. 73:915-920) and anthocyanins (Racchi and Manzocchi (1988) Plant Cell Rep. 7:78-81). However, the cultured cells displayed no apparent histological organization, preventing their use for cell-cell communication and cell fate determination studies. A suspension culture necessarily contains a mixture of cells: young meristematic cells, older cells and dead cells. Felker ((1987) Am. J. of Bot. 74:1912-19200) indicated that the cells in the in vitro endosperm suspension culture system are randomly distributed, with islands of specific cell types arising without any spatial organization. In contrast to the disorganized nature of suspension cultures, in intact endosperm, lipids are found mainly in the aleurone layer, protein and starch mainly in the starchy endosperm. Spatial disorganization in the cultures may result from irregular meritsematic activity, differential rates of cell growth throughout a tissue piece, or random breakage of tissue lumps, all caused by a lack of the control mechanisms that lead to the organized endosperm in planta.

Thus, additional in vitro culture methods are needed for studying long-term endosperm development and the interactions between both the endosperm cell types and the embryo. Of particular interest are in vitro culture methods that do not depend on the presence of maternal tissues and/or the use of complex culture media containing feeder cell suspensions, while maintaining the organizational intactness of the endosperm and the endosperm-embryo interface.

### SUMMARY OF THE INVENTION

It is an object of the present invention to provide methods for the in vitro culture of cereal endosperm. The methods of the present invention allow for the extended growth and development of the tissues of the isolated embryo sac, particularly the endosperm therein. Furthermore, the methods allow the endosperm to enter into a proliferative phase in which endosperm cells divide mitotically and differentiate to produce starchy endosperm and aleurone cells so as to produce the type of organization that is known to occur in endosperm that is grown in planta. Like in planta produced endosperm, the endosperms produced by the methods of the present invention are differentiated and comprises a single surface layer of aleurone cells and an interior mass of starchy endosperm cells. Such in planta-like organization is not known to occur in cereal endosperm that is produced by existing in vitro culture methods.

The present invention provides in vitro methods for culturing cereal endosperm. The methods comprise isolating a fertilized embryo sac from a cereal plant. The methods of the invention further involve exposing the isolated fertilized embryo sac or portion thereof to a proliferation medium for an extended period of time so as to promote the growth and development of the endosperm or the growth and development of both the endosperm and the embryo. The isolated fertilized embryo sac or portion thereof that is exposed to the proliferation medium comprises endosperm tissue or can alternatively comprise endosperm tissue and the embryo. The proliferation medium is suitable for promoting the in vitro growth and development of the endosperm or both the endosperm and the embryo.

In an embodiment of the invention, the methods for culturing endosperm from a cereal plant further involve removing the embryo from an isolated fertilized cereal embryo sac. The embryo can be separated from the endosperm after the fertilized embryo sac is isolated and prior to placing the endosperm in culture. Alternatively, the embryo can be removed after the embryo sac has been placed in contact with a culture medium. The methods further involve exposing the remaining portion of the embryo sac, particularly endosperm, to a proliferation medium so as to promote endosperm development in culture.

In another embodiment of the invention, cereal endosperm, particularly maize endosperm, that is cultured by the methods of the present invention produces bulges on its surface. These bulges are also referred to herein as “mini-endosperm” and begin to appear after about 10 days in culture. Such a mini-endosperm comprises an aleurone cell layer surrounding an interior mass of starchy endosperm cells. This type of organization is analogous to the organization of in planta produced endosperm.

The present invention provides methods for modulating the level of a polypeptide of interest in a cereal embryo sac or portion thereof. Such methods comprise introducing into a cultured cereal embryo sac or portion thereof a polynucleotide of interest, wherein the embryo sac or portion thereof is cultured by the methods of the present invention. In one embodiment of the invention, the polynucleotide of the polynucleotide of interest comprises a hairpin construct suitable for decreasing the level of said polypeptide of polypeptide of interest by RNAi. In another embodiment, the polynucleotide of interest is encoded by a transgene that is introduced into said embryo sac or portion thereof before, at the same time as, or after the polynucleotide of interest is introduced into said embryo sac or portion thereof. Additionally provided are transformed plant tissues and plant cells that comprise stably integrated in their genomes the polynucleotide of interest.

The present invention provides methods for determining the effect of a chemical of interest on endosperm development: Such methods make use of endosperm cells that are cultured by the methods disclosed herein. The methods for determining the effect of a chemical of interest on endosperm development comprise contacting an embryo sac or portion thereof with a chemical of interest, wherein said embryo sac or portion thereof is cultured by the methods of the present invention. The methods further involve monitoring the development of said endosperm in said embryo sac or portion thereof. In certain embodiments, the methods comprise the use of embryo sacs or portions thereof that have been transformed with a marker gene, particularly a marker gene that encodes a fluorescent protein. In such embodiments, the marker gene is operably linked to a promoter that drives expression in the endosperm or part thereof.

The present invention further provides methods for determining the subcellular localization of a protein in endosperm cells. Such methods involve the use of endosperm cells that are cultured by the methods disclosed herein. The methods for determining the subcellular localization of a protein in endosperm cells comprise introducing into an endosperm cell a nucleic acid construct comprising a polynucleotide encoding a protein of interest. Such a polynucleotide is operably linked to a promoter that drives expression in an endosperm cell. The polynucleotide is also operably linked to a marker gene encoding a fluorescent protein or functional portions thereof so as to allow for the production of a fusion protein comprising said protein of interest and said fluorescent protein or functional part thereof in a transformed embryo sac cell, particularly in an endosperm cell therein. The methods further involve determining the subcellular localization of the protein in the endosperm by detecting the subcellular location of fluorescence from said fluorescent protein. In one embodiment, determining the subcellular location of the protein involves microscopy, particular confocal microscopy.

The present invention further provides methods for culturing mini-endosperm. The methods comprise isolating mini-endosperm and exposing the mini-endosperm or portion thereof to a proliferation medium so as to promote the growth and development of the mini-endosperm. Such mini-endosperms can be isolated by excising or separating the mini-endosperms from, for example, existing in vitro cultured endosperm of the present invention.

Additionally provided are in vitro cultured embryo sacs and parts and cells thereof as well as that are produced by the methods of the present invention. Further provided are mini-endosperms and parts and cells thereof that are produced by the methods of the present invention.

### DETAILED DESCRIPTION OF THE INVENTION

The present invention is directed to methods for the in vitro culture of plant tissues, particularly plant tissues from cereal plants. In particular, the invention is directed to the in vitro culture of cereal endosperm. The cultures are initiated from isolated fertilized cereal embryo sacs or isolated parts of fertilized cereal embryo sacs that comprise endosperm. Such fertilized cereal embryo sacs or portions thereof are isolated after pollination of the flowers in which they occur in the cereal plant. The present invention is based on the discovery that a fertilized maize embryo sac can be manually isolated at an early developmental stage and that, when placed on either on a solid proliferation medium of the invention or in liquid proliferation medium of the invention, the embryo sac or the endosperm therein will continue to grow and develop in a manner that results in organization that is similar to in planta organization, particularly a single surface layer of aleurone cells and interior cell masses of starchy endosperm in the case of wild-type cereal plants. Furthermore, unlike the media used in some existing methods for culturing cereal embryo sacs or portions thereof, the proliferation media of the present invention do not depend on the use of feeder suspension cells, thereby eliminating any influence that such feeder cells have on the in vitro growth and development of the embryo sac and tissues contained therein, particularly the endosperm.

The present invention provides in vitro culture methods for cereal endosperm derived from isolated fertilized cereal embryo sacs or isolated portions or parts of fertilized cereal embryo sacs, particularly the endosperm. Such methods are suitable for the extended growth of isolated embryo sacs or portions thereof, allowing the endosperm to enter into a proliferative phase in which both starchy endosperm and aleurone cells divide mitotically. In particular, the methods of the present invention allow for the growth and development of the isolated fertilized cereal embryo sac or the endosperm therefrom for an extended period of time of at least about 5, 10, 15, or 20 days or even longer. Similar to in planta grown endosperm, the endosperm cultured by the methods disclosed herein stops growing and becomes necrotic, which is consistent with the cultured endosperm undergoing a similar genetic/developmental program as in planta grown endosperm. Thus, the methods of the present invention find use in producing tissues of the embryo sac, particularly the endosperm, that are suitable for use in studies of the development, genetics, cell biology, physiology, and biochemistry. In addition, the methods of the present invention find further use in producing plant tissues for use in studies of interactions between the tissues of the embryo sac, particularly the interactions between the endosperm and the embryo and the aleurone and starchy endosperm cells. It is recognized that the embryo sac tissues that are cultured by the methods disclosed herein are suitable for use in studying the development, genetics, cell biology, physiology, and biochemistry of one or more of the interacting tissues.

Unlike previously reported methods for culturing cereal embryo sacs or endosperm, the cells of embryo sac tissues that are produced by the present invention have the same type of organization as such tissues do in planta. In particular, the cultured endosperm of the present invention has the same type of organization as that of endosperm grown in planta. Typically, the in vitro cultured endosperm of the present invention is of a smaller total volume than in planta grown endosperm. However, the methods of the present invention are not limited to the production of in vitro cultured endosperm of a particular volume. Accordingly, in vitro cultured endosperm produced by the methods of the present invention can comprise a total volume that is smaller, larger, or about the same as the total volume of similar endosperm that is produced in planta.

While the present invention is not bound by any particular biologicial mechanism, the observed smaller volume of the in vitro cultured endosperm of the present invention may be due to a deficiency in the influx of carbon into the in vitro cultured endosperm as compared to in planta grown endosperm which benefits from its highly effective placento-chalazal transfer cell complex for solute transfer from source to sink tissues. In in vitro cultures that are initiated without removing the embryo, the embryos develop and precociously germinate around 20-25 DIV. We do not observe obvious differences in the morphology of in vitro endosperm grown with or without the embryo attached.

Like in planta grown endosperm, the cultured endosperm of the present invention comprises a single surface layer of aleurone cells and interior cell masses of starchy endosperm. The organization of the endosperm that is produced by the methods disclosed here is a distinguishing feature from endosperm produced by the in vitro culture method of Shannon ((1982) “Maize endosperm cultures”, in Sheridan, W. F., ed., Maize for Biological Research, Plant Molecular Biology Association, Charlottesville, Va., pp 397-400), which is not known to produce endosperm with an “in planta-like” organization.

After being exposed to proliferation medium for a period of time typically about 10 days for embryo sacs harvested at 6 DAP the in vitro cultured cereal endosperm of the present invention, particularly maize endosperm, differentiates so as to form bulges or projections on its surface. These bulges are also referred to herein as mini-endosperm because the organization of these bulges is analogous to endosperm with an aleurone cell layer surrounding a mass of starchy endosperm cells. However, in mini-endosperm from dek1 mutants, this aleurone cell layer may be lacking or not fully differentiated as is described more in detail in Example 10. Such mini-endosperms have been not reported previously to occur in cultured cereal embryo sac or endosperm tissues nor are they known to form in planta in wild-type endosperm. Thus, the methods of the present invention find further use in producing mini-endosperm from cereal endosperm.

It is recognized, however, that for mini-endosperm developing from in vitro grown dek1 endosperm, the aleurone cell layer can be absent or not fully differentiated as is described more in detail in Example 10.

While such bulges or mini-endosperms are not known to occur in planta in wild-type endosperm, Olsen ((2004) Maydica 49: 37-40) reported observing in defective maize kernel mutants the presence of spherical bodies of endosperm that are similar to the mini-endosperms produced by the in vitro culture methods of the present invention. Like the mini-endosperms of the present invention, the spherical bodies of endosperm comprised an interior mass of starchy endosperm cells and are covered by at least one layer of aleurone cells. The spherical bodies were observed, however, on the interior endosperm cavity of the in planta grown mutant endosperms. It was not known at the time when these observations were published whether spherical bodies of endosperm formed because of a developmental mutation in these mutants, nor was it known whether the organization into these mini-endosperm structures required maternal factors from the surrounding seed tissues.

Thus, the methods of the present invention find use in studying in vitro development of the embryo sac and the cells and tissues contained therein, including, but not limited to, transfer cells, starchy endosperm cells, aleurone cells, and mini-endosperm and any of the cells and tissues comprising the mini-endosperm. In particular, the methods of the invention find use in studying the growth and development of the endosperm and the cells and tissues therein. Within a few days after pollination, the most abundant tissue of a fertilized cereal embryo sac is the endosperm. The maize endosperm consists of four cell types, the starchy endosperm, the aleurone layer, the transfer cells and the cells of the embryo surrounding region. See also, Becraft et al. (2000) Developmental biology of endosperm development, Kluwer Academic Publ., Dordrecht, N L; Olsen (2001) Annu. Rev. Plant Physiol. Plant Mol. Biol. 52: 233-267; both of which are herein incorporated by reference.

In the description that follows, a number of terms are used extensively. The following definitions are provided to facilitate understanding of the invention.

An embryo sac is typically an eight-nucleate female gametophyte. The embryo sac arises from the megaspore by successive mitotic divisions.

A megaspore is one of the four haploid spores originating from the meiotic division of the diploid megaspore mother cell in the ovary and which gives rise to the megagametophyte.

A microspore is one of the four haploid spores originating from the meiotic division of the diploid microspore mother cell in the anther and which gives rise to the pollen grain.

The central cell of the embryo sac comprises two nuclei that are known as polar nuclei.

The polar nuclei are two centrally located nuclei in the embryo sac that unite with the nucleus of a sperm cell in a triple fusion. In certain seeds, such as, for example, cereal seeds, the product of this triple fusion develops into the 3n endosperm.

A fertilized embryo sac is an embryo sac following the fusion of a sperm cell with the egg cell and/or the fusion of a sperm cell with the central cell. Typically, a fertilized embryo sac results from double fertilization, wherein a first sperm cell fuses with the egg cell and a second sperm cell fuses with the central cell.

An ovule is the structure in seed plants containing the female gametophyte. The ovule is comprised of the nucellus which is surrounded by one or two integuments, and it is attached to the placenta by a stalk know as the funiculus.

Nucellus is the tissue within the ovule in which the female gametophyte (i.e., the embryo sac) develops. The nucellus is the maternal tissue that is adjacent to the embryo sac.

The methods of the invention involve the isolation of fertilized cereal embryo sacs. Because the embryo sac is found within the ovule, the methods of the invention involve removing the fertilized embryo sac from the ovule. The methods of the invention do not depend on a particular method for removing the embryo sac. Methods for removing the fertilized embryo sac from the ovule include, for example, the manual isolation of the embryo sac with dissecting or surgical tools. Generally such tools are suitable for the microdissection of plant tissues and include, but not limited to, forceps, a scalpel, a knife, scissors, dissecting needle, a corneal knife, and the like. Typically, such tools are sterilized prior to use. In one embodiment of the invention, forceps are used to isolate the fertilized embryo sacs by dissecting the embryo sacs from intact ovules.

In one embodiment of the invention, cereal embryo sacs are isolated from maize kernels. Pollinated ears are harvested from maize plants at, for example 6 DAP, and the harvested ears sprayed with 70% (v/v) ethanol and allowed to incubate for 5 minutes to surface sterilize the plant tissue. The kernels are then dissected under a dissecting microscope. Using fine forceps to hold the kernel straight up (i.e., tip up and basal end down), a small cut, about one-fourth to one-third of the length of the kernel from the top, is made with a scalpel to divide the kernel tip into two parts that remain attached to each other. The kernel is then opened up by holding one part of the kernel with forceps and pushing the other part away from the other. The embryo sac is then gently removed with the fine forceps and placed on solid proliferation medium or in liquid proliferation medium.

In one embodiment of the invention, embryo sacs are isolated from the ovules of maize plants. When the embryo sacs are manually isolated from maize ovules by dissection from the ovule at 3 DAP or later, the fertilized embryo sacs retain little or no adhering maternal tissue (e.g., nucellus), resulting in embryo sacs that are substantially free of maternal tissue. Such embryo sacs are distinguished from the embryo sacs of the nucellus slab culture system of Laurie et al. ((1999) In Vitro Cell Dev. Biol.—Plant 35:320-325), which are surrounded by tissue from the ovary wall and nucellus. However, when younger embryo sacs (i.e., 0, 1, or 2 DAP) are isolated, substantial amounts of nucellus tissue typically adheres to the embryo sac. Nevertheless, such younger embryo sacs are not surrounded by tissue from the ovary wall, unlike the embryo sacs of the nucellus slab culture system of Laurie et al. which are surrounded by tissue from both the ovary wall and nucellus ((1999) In Vitro Cell Dev. Biol.—Plant 35:320-325). While the methods of the present invention are not bound by any particular biological mechanism, it is recognized that prior to about 3 DAP, the nucellus adheres tightly to the outer surface of the embryo sac, and that by about 3 DAP, the embryo sac floats freely in nucellus lysate. Furthermore, it is believed that such nucellus lysate results from the autolysis of the nucellus parenchyma cells next to the embryo sac undergo prior to about 3 DAP.

In certain embodiments of the invention, the methods for culturing cereal endosperm can further optionally comprise removing the embryo from a fertilized embryo sac that is isolated as described herein. The embryo can be removed from the isolated embryo sac either before or after the isolated fertilized embryo sac is exposed to proliferation medium. Typically, the methods involve the manual separation or removal of the embryo from the remainder of the embryo sac or endosperm with dissecting or surgical tools. Generally such tools are suitable for the microdissection of plant tissues and include, but not limited to, forceps, a scalpel, a knife, scissors, dissecting needle, a corneal knife, and the like. Typically, such tools will have been sterilized prior to use. In one embodiment of the invention, forceps are used to remove the embryo from the embryo sac.

The invention involves the use of fertilized embryo sacs. Generally, the methods of the invention involve the use of any intact fertilized embryo sacs that can be isolated from ovules. In one embodiment of the invention, fertilized embryo sacs are isolated from maize ovules on the same day as pollination or 1, 2, 3, 4, 5, 6, 7, 8, 9, 10, 11, 12, 13, 14, 15 or more days after pollination (DAP). Generally, it is difficult to separate the fertilized maize embryo sacs from adhering nucellus tissue any earlier than 3 DAP. Optimally, the fertilized embryo sacs are isolated from maize ovules at 3 to 12 days after pollination. More optimally, the fertilized embryo sacs are isolated from maize ovules at 4 to 8 days after pollination. Even more optimally, the fertilized embryo sacs are isolated from maize ovules at 5 to 7 days after pollination. Most optimally, the fertilized embryo sacs are isolated from maize ovules at 6 days after pollination.

In certain embodiments of the invention, the methods involve the use of embryo sacs or portions of embryo sacs that are substantially free of maternal tissue. Generally, in such embodiments, an embryo sac of the invention is isolated from maternal tissue prior to being exposed to proliferation medium and are comprised of less than about 50% by volume maternal tissue. Optimally, such an isolated embryo sac is comprised of less than about 40%, 30%, 25%, 20%, 15%, 10%, 5% or less by volume maternal tissue. In some other embodiments of the invention, an isolated embryo sac of the invention is comprised of less than about 0.5% 0.2%, or 0.1% by volume maternal tissue or is free of maternal tissue.

The methods of the present invention involve exposing a fertilized cereal embryo sac or portion thereof to a proliferation medium. The proliferation media of the invention are plant culture media that are generally suitable for the culturing of embryo sacs and/or portions thereof so as to promote the growth and development of in vitro cultured plant tissues. In particular, a proliferation medium of the invention promotes endosperm development or both endosperm and embryo development. In certain embodiments of the invention, the proliferation media can also promote the growth and development of isolated mini-endosperms.

Although the methods of the present invention do not depend on a particular proliferation medium comprising particular components, it is recognized that a proliferation medium of the invention will generally comprise effective amounts of a basal salt mixture and a carbon source. A proliferation medium of the invention optionally comprises an effective amount of one or more of the following components: a phytohormone, a vitamin mixture, thiamine, asparagine, and a gelling agent.

By “effective amount” is intended an amount of an agent such as, for example, a phytohormone, or other component of a culture medium of the invention that, when present in a culture medium of the invention, is capable of causing the desired effect in culture on a plant or part thereof including, but not limited to, embryo sacs, endosperm, embryos, and isolated plant cells from these and other plant tissues, tissues, organs and whole plants, seeds, and callus. It is recognized that an “effective amount” may vary depending on factors, such as, for example, the genotype of the plant, the target tissue, the method of preparation of the culture medium, other components in the medium, temperature, light, relative humidity, pH, and the like. Further, it is recognized that an “effective amount” of a particular agent or component can be determined by administering a range of amounts of the agent in culture to the plant or part thereof and then determining which amount or amounts cause the desired effect.

Typically, plant culture media of the invention will generally comprise a basal salt mixture. Such basal salt mixtures are known in the art and include, but are not limited to, Murashige & Skoog (MS), N6, NB, Gamborg's, Linsmaier & Skoog, Nitsch & Nitsch and the like. Generally, the pH of the plant culture media of the invention will fall within the range of about pH 4 to about pH 7, optimally between about pH 5.5 and about pH 6.5, more optimally between about pH 5.6 and pH 6.0, most optimally pH 5.8.

The plant culture media of the invention can optionally comprise a vitamin mixture or a combination of one or more of such vitamin mixtures. Such vitamin mixtures are known in the art and include, but are not limited to, MS vitamins, Gamborg's vitamins, MEM vitamins, Schenk and Hildebrandt vitamins, Nitsch & Nitsch vitamins, N6 vitamins, and the like.

Plant culture media of the invention can additionally comprise a carbon source. Typically, the carbon source is a form of reduced carbon such as, for example, sucrose. The methods of the invention do not depend on a particular carbon source, only that the carbon source may be metabolized cereal cells or tissues thereof in culture. In addition to sucrose, carbon sources include, but are not limited to, maltose, glucose, fructose, galactose, raffinose, stachyose, mannitol, sorbitol, and mixtures thereof.

In certain embodiments of the invention, the proliferation media of the invention comprise a high concentration of carbon source. In particular, such proliferation media comprise a high concentration of sucrose or maltose, particularly an effective concentration of at least about 10% (w/v). Optimally, the proliferation medium comprises about 10, 11, 12, 13, 14, 15, 16, 17, 18, 19, or 20% (w/v) sucrose or maltose. More optimally, the proliferation medium comprises an effective concentration of sucrose (w/v) in the range of about 12% to about 18%. Even more optimally, the proliferation medium comprises an effective concentration of sucrose (w/v) in the range of about 14% to about 16%. Most optimally, the proliferation medium comprises about 15% (w/v) sucrose.

The phytohormones or plant growth regulators of the invention include, but are not limited to, both free and conjugated forms of naturally occurring phytohormones or plant growth regulators. Additionally, the phytohormones of the invention encompass synthetic analogues, inhibitors of the synthesis, degradation, conjugation, transport, binding or action, precursors of such naturally occurring phytohormones and any other compounds that are known to have a phytohormone-like effect on the growth and development of plants. Phytohormones include, but are not limited to auxins, cytokinins, abscisic acid, gibberellins and ethylene, and conjugates, synthetic analogues, inhibitors and precursors thereof.

Naturally occurring cytokinins and synthetic analogues of cytokinins include, but are not limited to, kinetin, zeatin, zeatin riboside, zeatin riboside phosphate, dihydrozeatin, isopentyl adenine, 6-benzylaminopurine (BAP) and thidiazuron (TDZ), or mixture thereof.

In certain embodiments of the invention, the proliferation medium comprises an effective concentration of at least one cytokinin. In such embodiments, the effective concentration of cytokinin in the proliferation medium is generally at least about 0.001, 0.005, 0.0025, 0.010, 0.02, 0.025, 0.03, 0.04, 0.05, 0.06, 0.07, 0.075, 0.08, 0.09, or 0.1 mg/mL. Typically, the effective concentration of cytokinin is less than about 10, 5, 1, 0.9, 0.8, 0.75, 0.7, 0.6, 0.5, 0.4, 0.3, 0.25, 0.2, or 0.15 mg/mL. Optimally, the effective concentration of cytokinin is in the range of about 0.001 mg/mL to about 10 mg/mL. More optimally, the effective concentration of cytokinin is in the range of about 0.01 mg/mL to about 1 mg/mL. Even more optimally, the effective concentration of cytokinin is in the range of about 0.025 mg/mL to about 0.5 mg/mL. Still even more optimally, the effective concentration of cytokinin is in the range of about 0.05 mg/mL to about 0.25 mg/mL. Yet still even more optimally, the effective concentration of cytokinin is in the range of about 0.075 mg/mL to about 0.125 mg/mL. Most optimally, the effective concentration of cytokinin is 0.1 mg/mL.

Naturally occurring auxins and synthetic analogues of auxins include, but are not limited to, indoleacetic acid (IAA), 3-indolebutyric acid (IBA), α-napthaleneacetic acid (NAA), 2,4-dichlorophenoxyacetic acid (2,4-D), 4-(2,4-dichlorophenoxy) butyric acid, 2,4,5-trichlorophenoxyacetic acid (2,4,5-T), (4-chloro-2-methylphenoxy)acetic acid (MCPA), 4-(4-chloro-2-methylphenoxy) butanoic acid (MCPB), mecoprop, dicloprop, quinclorac, picloram, triclopyr, clopyralid, fluroxypyr, dicamba, and mixtures thereof.

Inhibitors of auxins include, but are not limited, inhibitors of enzymes in the biosynthesis pathway leading to the formation of an auxin in a plant and auxin transport inhibitors, such as, for example, 3,4,5-triiodobenzoic acid (TIBA), naphthylphthalamic acid, 9-hydroxyfluorene-9-carboxylic acid, and mixtures thereof. Inhibitors of ABA biosynthesis include, but are not limited to, norflurazon. Inhibitors of ethylene include, but are not limited to, inhibitors of ethylene synthesis or evolution such as, for example, aminoethoxyvinylglycine (AVG) and silver ions, and inhibitors of ethylene action such as, for example, 2,5-norbornadiene, and mixtures of any two or more of such ethylene inhibitors.

The plant culture media of the invention may additionally comprise other components known in the art such as, for example, vitamins, co-factors, micronutrients, charcoal, trace elements, myo-inositol, amino acids and the like. Solid plant culture media of the invention additionally comprise a solidifying agent such as, for example, agar. The plant culture media of the invention may also be adapted for use in transformation methods and may additionally comprise selective agents, such as, for example, antibiotics and herbicides. Such selective agents are known in the art and include, but are not limited to, kanamycin, geneticin, cefotaxime, carbenicillin, hygromycin, glyphosate, glufosinate or phosphinothricin, bialaphos, chlorsulfuron, bromoxynil, imidazolinones, 2,4-D, methotrexate, and mixtures thereof.

In one embodiment of the invention, the proliferation medium is a modified MS medium comprising 0.4 mg/L L-asparagine, 0.1 mg/mL 6-benzylaminopurine (BAP) and 15% sucrose, pH 5.8 as described in U.S. Pat. No. 6,300,543; herein incorporated by reference. If solid medium is desired, the proliferation medium can further comprise an effective concentration of a gelling agent such as, for example, agar, phytagel, gelrite, agarose, and mixtures thereof. Optimally, solid proliferation media of the invention comprise about 3 g/L gelrite.

The methods of the invention involve exposing plant tissues to a proliferation medium and/or other plant culture media. By “exposing” is intended placing the tissue in the vicinity of the medium wherein at least one component of the medium is able to enter the tissue. Typically, the tissue is exposed to the medium by placing the tissue in direct contact with a solid, semisolid, or liquid medium. It is recognized, however, that the tissue can be exposed to the medium without directly contacting the medium. For example, the tissue can be exposed to a medium by placing the tissue on a filter-paper-lined surface of a plate of solid medium.

It is also recognized that the plant tissues of the invention which are exposed to a particular plant culture media may be routinely transferred to fresh plant culture media when necessary. Such routine transfers of plant tissue to fresh plant culture media are known in the art. Generally, endosperm cultured by the methods disclosed herein need not be transferred to fresh medium during the lifespan of the cultured endosperm as necrosis of the cultured endosperm typically occurs.

Furthermore, it is recognized that the mini-endosperms or bulges that develop on the in vitro cultured cereal endosperm of the present invention may be excised or separated from the in vitro cultured endosperm to which they are attached and placed on proliferation medium for further propagation. The present invention does not depend on cereal endosperm of a particular genotype as the source of mini-endosperm, only that such endosperm produces mini-endosperms or bulges as disclosed herein. Such genotypes include, but are limited to, wild-type and endosperm mutants such as, for example, the maize endosperm mutants, dek1, sal1, and crinkly4 (cr4) (Becraft et al. (1996) Science 273:1406-1409). Additionally, such endosperm may have been transformed directly or be from a transgenic plant, particularly a stably transformed, transgenic plant. Generally, such mini-endosperms can be isolated by excising or separating the mini-endosperms from existing in vitro cultured endosperm or even from in planta grown endosperm with, for example, a scalpel or forceps, and then placed on fresh proliferation medium for further growth and development. Accordingly, the present invention additionally provides methods for culturing mini-endosperm comprising the steps of isolating mini-endosperm or portion thereof and exposing the mini-endosperm or portion thereof to a proliferation medium so as to promote the growth and development of the mini-endosperm or portion thereof.

The methods of the invention involve exposing an embryo sac or part thereof to a proliferation medium under environmental conditions that are favorable to the growth and development of the endosperm or both the endosperm and embryo. Such environmental conditions include, for example, temperature, whether constant or varied throughout a given day, (e.g., higher temperature in the light period and lower in the dark period), photoperiod, light intensity, complete darkness, relative humidity, and the like. The invention does not depend on any particular combination of such environmental conditions, only that such combination of conditions favor the growth and development of the endosperm or both the endosperm and embryo on proliferation medium. In an embodiment of the invention, isolated maize embryo sacs or portions thereof, particularly endosperm, are exposed to a liquid or solid proliferation medium in complete darkness at 30° C. When liquid proliferation medium is used in this particular embodiment of the invention, an individual embryo sac or portion thereof endosperm is placed in a vessel such as, for example, the well of a 96-well microtiter plate with a suitable amount of proliferation medium. Such an embryo sac or portion thereof, may be, but need not be, shaken while exposed to liquid proliferation medium.

The methods of the invention involve exposing embryo sacs or portion thereof, particularly endosperm, to a proliferation medium for an extended period of time. Typically, such an extended period of time is at least 5 days, although in certain embodiments of the extended period of time comprises 1, 2, 3, or 4 days. In other embodiments, the embryo sacs or portions thereof are exposed a proliferation medium for an extended period of time comprising at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, or more days. Optimally, the embryo sacs are exposed to proliferation medium for about 5 to 25 days. More optimally, the embryo sacs are exposed to proliferation medium for about 10 to 20 days.

The methods of the invention do not depend on particular plant culture media. Any suitable plant culture medium known in the art may be employed in the methods of the present invention. For a general description of plant culture media and basic techniques in plant cell, tissue and organ culture, see Evans et al. eds. (1983), Handbook of Plant Cell Culture, Vol. 1: Techniques for Propagation and Breeding (MacMillan, London); Sharp et al eds. (1984) Handbook of Plant Cell Culture, Vol. 2: Crop Species (MacMillan, London); Ammirato et al. eds. (1984) Handbook of Plant Cell Culture, Vol. 3: Crop Species (MacMillan, London); and Evans et al. eds. (1983) Handbook of Plant Cell Culture, Vol. 4: Techniques and Applications (MacMillan, London); all of which are herein incorporated by reference.

The methods of the invention involve the use of fertilized embryo sacs that are isolated from cereal plants. Such cereal plants can be grown, for example, in the field, greenhouse, or growth chamber. It is recognized that environmental conditions, such as, for example, temperature, light levels, photoperiod, relative humidity, and the like can be controlled when the plants are grown in a greenhouse or growth chamber. While the methods of the present invention are not known to depend on cereal embryo sacs from plants that are grown under any particular environmental conditions, the cereal plants of the invention are typically grown under environmental conditions that are favorable for the growth and development of cereal plants, particularly those environmental conditions that are favorable for the reproductive growth and development of cereal plants. It is recognized that those of ordinary skill in the art either know or know how to determine such favorable conditions and further that such favorable conditions can vary from one species of cereal plant to another or even from one genotype to another within a single cereal plant species.

A cereal plant of the invention can be pollinated by any method known in the art including, but not limited to, method involving manual pollination of the cereal plant with its own pollen or pollen from another cereal plant of the same genotype or a different genotype and also methods involving the use of male sterility in the pollen receptor plant. Alternatively, a cereal plant of the invention can be allowed to self-pollinate or can be allowed to pollinate with pollen from an adjacent cereal plant.

In one embodiment of the invention, maize plants that are used as the source of embryo sacs are grown in a greenhouse with the temperature maintained between a high temperature of about 82° F. and a low temperature of about 68-70° F. The photoperiod is comprised of a light period of about 15-16 hours and a dark period of 9 or 10 hours, respectively. The photosynthetically active radiation is generally maintained in the vicinity of at least about 1800 PAR. The plants are grown in pots filled with a standard commercial potting medium, such as, for example, Metro-Mix 700 (The Scotts Company, Marysville, Ohio, USA) and fertilized as needed with a standard fertilizer mixture, such as, for example, 20-10-20 (N-P-K).

The invention further provides cultured plant tissues, particularly cultured cereal embryo sacs and cells thereof, cultured endosperm tissue and cells thereof, and cultured embryos and cells thereof. Such cultured plant tissues are suitable for use in both stable and transient transformation methods to introduce at least one polynucleotide of interest. Accordingly, the present invention encompasses such transformed plant parts, plant tissues, and plants cells that comprise the polynucleotide of interest.

The invention provides methods for introducing a polynucleotide or polypeptide of interest into at least one plant cell of a cultured fertilized embryo sac or portion thereof. Such methods involve the use of fertilized embryo sacs or portion or portions thereof that are produced by the culture methods disclosed herein. Such parts of fertilized embryo sacs include, for example, the endosperm and the embryo. The endosperm includes, for example, the aleurone layer, the starchy endosperm, transfer cells, and the embryo surrounding region.

The use of the term “polynucleotide” is not intended to limit the present invention to polynucleotides comprising DNA. Those of ordinary skill in the art will recognize that polynucleotides of interest can comprise ribonucleotides and combinations of ribonucleotides and deoxyribonucleotides. Such deoxyribonucleotides and ribonucleotides include both naturally occurring molecules and synthetic analogues. The polynucleotides of interest of the present invention also encompass all forms of sequences including, but not limited to, single-stranded forms, double-stranded forms, hairpins, stem-and-loop structures, and the like.

The polynucleotides of interest can be provided in expression cassettes for expression in the cereal plant of interest. The cassette will include 5′ and 3′ regulatory sequences operably linked to a polynucleotide of interest. “Operably linked” is intended to mean a functional linkage between two or more elements. For example, an operable linkage between a polynucleotide of interest and a regulatory sequence (i.e., a promoter) is functional link that allows for expression of the polynucleotide of interest. Operably linked elements may be contiguous or non-contiguous. When used to refer to the joining of two protein coding regions, by operably linked is intended that the coding regions are in the same reading frame. The cassette may additionally contain at least one additional gene to be cotransformed into the organism. Alternatively, the additional gene(s) can be provided on multiple expression cassettes. Such an expression cassette is provided with a plurality of restriction sites and/or recombination sites for insertion of the polynucleotide of interest to be under the transcriptional regulation of the regulatory regions. The expression cassette may additionally contain selectable marker genes.

The expression cassette will include in the 5′-3′ direction of transcription, a transcriptional and translational initiation region (i.e., a promoter), a polynucleotide of interest, and a transcriptional and translational termination region (i.e., termination region) functional in grain plants. The regulatory regions (i.e., promoters, transcriptional regulatory regions, and translational termination regions) and/or the polynucleotide of interest may be native/analogous to the host cell or to each other. Alternatively, the regulatory regions and/or the polynucleotide of interest may be heterologous to the host cell or to each other. As used herein, “heterologous” in reference to a sequence is a sequence that originates from a foreign species, or, if from the same species, is substantially modified from its native form in composition and/or genomic locus by deliberate human intervention. For example, a promoter operably linked to a heterologous polynucleotide is from a species different from the species from which the polynucleotide was derived, or, if from the same/analogous species, one or both are substantially modified from their original form and/or genomic locus, or the promoter is not the native promoter for the operably linked polynucleotide. As used herein, a chimeric gene comprises a coding sequence operably linked to a transcription initiation region that is heterologous to the coding sequence.

While it may be optimal to express the sequences using heterologous promoters, the native promoter sequences may be used. Such constructs can change expression levels of a polypeptide of interest in the plant or plant cell. Thus, the phenotype of the plant or plant cell can be altered.

The termination region may be native with the transcriptional initiation region, may be native with the operably linked polynucleotide of interest, may be native with the plant host, or may be derived from another source (i.e., foreign or heterologous) to the promoter, the polynucleotide of interest, the plant host, or any combination thereof. Convenient termination regions are available from the Ti-plasmid of A. tumefaciens, such as the octopine synthase and nopaline synthase termination regions. See also Guerineau et al. (1991) Mol. Gen. Genet. 262:141-144; Proudfoot (1991) Cell 64:671-674; Sanfacon et al. (1991) Genes Dev. 5:141-149; Mogen et al. (1990) Plant Cell 2:1261-1272; Munroe et al. (1990) Gene 91:151-158; Ballas et al. (1989) Nucleic Acids Res. 17:7891-7903; and Joshi et al. (1987) Nucleic Acids Res. 15:9627-9639.

Where appropriate, the polynucleotides may be optimized for increased expression in the transformed plant. That is, the polynucleotides can be synthesized using plant-preferred codons for improved expression. See, for example, Campbell and Gowri (1990) Plant Physiol. 92:1-11 for a discussion of host-preferred codon usage. Methods are available in the art for synthesizing plant-preferred genes. See, for example, U.S. Pat. Nos. 5,380,831, and 5,436,391, and Murray et al. (1989) Nucleic Acids Res. 17:477-498, herein incorporated by reference.

Additional sequence modifications are known to enhance gene expression in a cellular host. These include elimination of sequences encoding spurious polyadenylation signals, exon-intron splice site signals, transposon-like repeats, and other such well-characterized sequences that may be deleterious to gene expression. The G-C content of the sequence may be adjusted to levels average for a given cellular host, as calculated by reference to known genes expressed in the host cell. When possible, the sequence is modified to avoid predicted hairpin secondary mRNA structures.

The expression cassettes may additionally contain 5′ leader sequences. Such leader sequences can act to enhance translation. Translation leaders are known in the art and include: picomavirus leaders, for example, EMCV leader (Encephalomyocarditis 5′ noncoding region) (Elroy-Stein et al. (1989) Proc. Natl. Acad. Sci. USA 86:6126-6130); potyvirus leaders, for example, TEV leader (Tobacco Etch Virus) (Gallie et al. (1995) Gene 165(2):233-238), MDMV leader (Maize Dwarf Mosaic Virus) (Virology 154:9-20), and human immunoglobulin heavy-chain binding protein (BiP) (Macejak et al. (1991) Nature 353:90-94); untranslated leader from the coat protein mRNA of alfalfa mosaic virus (AMV RNA 4) (Jobling et al. (1987) Nature 325:622-625); tobacco mosaic virus leader (TMV) (Gallie et al. (1989) in Molecular Biology of RNA, ed. Cech (Liss, New York), pp. 237-256); and maize chlorotic mottle virus leader (MCMV) (Lommel et al. (1991) Virology 81:382-385). See also, Della-Cioppa et al. (1987) Plant Physiol. 84:965-968.

In preparing the expression cassette, the various DNA fragments may be manipulated, so as to provide for the DNA sequences in the proper orientation and, as appropriate, in the proper reading frame. Toward this end, adapters or linkers may be employed to join the DNA fragments or other manipulations may be involved to provide for convenient restriction sites, removal of superfluous DNA, removal of restriction sites, or the like. For this purpose, in vitro mutagenesis, primer repair, restriction, annealing, resubstitutions, e.g., transitions and transversions, may be involved.

A number of promoters can be used in the practice of the invention, including the native promoter of the polynucleotide sequence of interest. The promoters can be selected based on the desired outcome. The nucleic acids can be combined with constitutive, tissue-preferred, or other promoters for expression in plants.

Such constitutive promoters include, for example, the core promoter of the Rsyn7 promoter and other constitutive promoters disclosed in WO 99/43838 and U.S. Pat. No. 6,072,050; the core CaMV 35S promoter (Odell et al. (1985) Nature 313:810-812); rice actin (McElroy et al. (1990) Plant Cell 2:163-171); ubiquitin (Christensen et al. (1989) Plant Mol. Biol. 12:619-632 and Christensen et al. (1992) Plant Mol. Biol. 18:675-689); pEMU (Last et al. (1991) Theor. Appl. Genet. 81:581-588); MAS (Velten et al. (1984) EMBO J. 3:2723-2730); ALS promoter (U.S. Pat. No. 5,659,026), and the like. Other constitutive promoters include, for example, U.S. Pat. Nos. 5,608,149; 5,608,144; 5,604,121; 5,569,597; 5,466,785; 5,399,680; 5,268,463; 5,608,142; and 6,177,611.

Chemical-regulated promoters can be used to modulate the expression of a gene in a plant through the application of an exogenous chemical regulator. Depending upon the objective, the promoter may be a chemical-inducible promoter, where application of the chemical induces gene expression, or a chemical-repressible promoter, where application of the chemical represses gene expression. Chemical-inducible promoters are known in the art and include, but are not limited to, the maize In2-2 promoter, which is activated by benzenesulfonamide herbicide safeners, the maize GST promoter, which is activated by hydrophobic electrophilic compounds that are used as pre-emergent herbicides, and the tobacco PR-1a promoter, which is activated by salicylic acid. Other chemical-regulated promoters of interest include steroid-responsive promoters (see, for example, the glucocorticoid-inducible promoter in Schena et al. (1991) Proc. Natl. Acad. Sci. USA 88:10421-10425 and McNellis et al. (1998) Plant J. 14(2):247-257) and tetracycline-inducible and tetracycline-repressible promoters (see, for example, Gatz et al. (1991) Mol. Gen. Genet. 227:229-237, and U.S. Pat. Nos. 5,814,618 and 5,789,156), herein incorporated by reference.

Tissue-preferred promoters can be utilized to target enhanced expression of a polynucleotide of interest within a particular plant tissue. Tissue-preferred promoters include Yamamoto et al. (1997) Plant J. 12(2):255-265; Kawamata et al. (1997) Plant Cell Physiol. 38(7):792-803; Hansen et al. (1997) Mol. Gen Genet. 254(3):337-343; Russell et al. (1997) Transgenic Res. 6(2):157-168; Rinehart et al. (1996) Plant Physiol. 112(3): 1331-1341; Van Camp et al. (1996) Plant Physiol. 112(2):525-535; Canevascini et al. (1996) Plant Physiol. 112(2):513-524; Yamamoto et al. (1994) Plant Cell Physiol. 35(5):773-778; Lam (1994) Results Probl. Cell Differ. 20:181-196; Orozco et al. (1993) Plant Mol. Biol. 23(6): 1129-1138; Matsuoka et al. (1993) Proc Natl. Acad. Sci. USA 90(20):9586-9590; and Guevara-Garcia et al. (1993) Plant J. 4(3):495-505. Such promoters can be modified, if necessary, for weak expression.

The expression cassette can also comprise a selectable marker gene for the selection of transformed cells. Selectable marker genes are utilized for the selection of transformed cells or tissues. Marker genes include genes encoding antibiotic resistance, such as those encoding neomycin phosphotransferase II (NEO) and hygromycin phosphotransferase (HPT), as well as genes conferring resistance to herbicidal compounds, such as glufosinate ammonium, bromoxynil, imidazolinones, and 2,4-dichlorophenoxyacetate (2,4-D). Additional selectable markers include phenotypic markers such as β-galactosidase and fluorescent proteins such as Zoanthus sp. yellow fluorescent protein (ZsYellow) that has been engineered for brighter fluorescence (Matz et al. (1999) Nature Biotech. 17:969-973; available from BD Biosciences Clontech, Palo Alto, Calif., USA, catalog no. K6100-1), green fluorescent protein (GFP) (Su et al. (2004) Biotechnol Bioeng 85:610-9 and Fetter et al. (2004) Plant Cell 16:215-28), cyan florescent protein (CYP) (Bolte et al. (2004) J. Cell Science 117:943-54 and Kato et al. (2002) Plant Physiol 129:913-42), and yellow florescent protein (PhiYFP™ from Evrogen, see, Bolte et al. (2004) J. Cell Science 117:943-54). For additional selectable markers, see generally, Yarranton (1992) Curr. Opin. Biotech. 3:506-511; Christopherson et al. (1992) Proc. Natl. Acad. Sci. USA 89:6314-6318; Yao et al. (1992) Cell 71:63-72; Reznikoff (1992) Mol. Microbiol. 6:2419-2422; Barkley et al. (1980) in The Operon, pp. 177-220; Hu et al. (1987) Cell 48:555-566; Brown et al. (1987) Cell 49:603-612; Figge et al. (1988) Cell 52:713-722; Deuschle et al. (1989) Proc. Natl. Acad. Aci. USA 86:5400-5404; Fuerst et al. (1989) Proc. Natl. Acad. Sci. USA 86:2549-2553; Deuschle et al. (1990) Science 248:480-483; Gossen (1993) Ph.D. Thesis, University of Heidelberg; Reines et al. (1993) Proc. Natl. Acad. Sci. USA 90:1917-1921; Labow et al. (1990) Mol. Cell. Biol. 10:3343-3356; Zambretti et al. (1992) Proc. Natl. Acad. Sci. USA 89:3952-3956; Baim et al. (1991) Proc. Natl. Acad. Sci. USA 88:5072-5076; Wyborski et al. (1991) Nucleic Acids Res. 19:4647-4653; Hillenand-Wissman (1989) Topics Mol. Struc. Biol. 10: 143-162; Degenkolb et al (1991) Antimicrob. Agents Chemother. 35:1591-1595; Kleinschnidt et al. (1988) Biochemistry 27:1094-1104; Bonin (1993) Ph.D. Thesis, University of Heidelberg; Gossen et al. (1992) Proc. Natl. Acad. Sci. USA 89:5547-5551; Oliva et al. (1992) Antimicrob. Agents Chemother. 36:913-919; Hlavka et al. (1985) Handbook of Experimental Pharmacology, Vol. 78 (Springer-Verlag, Berlin); Gill et al. (1988) Nature 334:721-724. Such disclosures are herein incorporated by reference.

Genes encoding other fluorescent protein that can be used in the methods of the present invention include, not limited to, those disclosed in WO 00/34321 (e.g., cFP484), WO 00/34526 (e.g., drFP583), WO 00/34323 (e.g., dgFP512), WO 00/34322 (e.g., dsFP483), WO 00/34318 (e.g., zFP506), WO 00/34319 (e.g., asFP600), WO 00/34320 (e.g., amFP486, ZsCyan), WO 00/34325 (e.g., zFP538), WO 00/34326 (e.g., drFP583), and WO 00/34324 (e.g., FP592); the disclosures of which are herein incorporated by reference in their entirety.

The above list of selectable marker genes is not meant to be limiting. Any selectable marker gene can be used in the present invention.

The methods of the invention involve introducing a polynucleotide or polypeptide into a plant. “Introducing” is intended to mean presenting to the plant the polynucleotide or polypeptide in such a manner that the sequence gains access to the interior of a cell of the plant. The methods of the invention do not depend on a particular method for introducing a sequence into a plant, only that the polynucleotide or polypeptides gains access to the interior of at least one cell of the plant. Methods for introducing polynucleotide or polypeptides into plants are known in the art including, but not limited to, stable transformation methods, transient transformation methods, and virus-mediated methods.

“Stable transformation” is intended to mean that the nucleotide construct introduced into a plant integrates into the genome of the plant and is capable of being inherited by the progeny thereof. “Transient transformation” is intended to mean that a polynucleotide is introduced into the plant and does not integrate into the genome of the plant or a polypeptide is introduced into a plant.

Transformation protocols as well as protocols for introducing polypeptides or polynucleotide sequences into plants may vary depending on the type of plant or plant cell, i.e., monocot or dicot, targeted for transformation. Suitable methods of introducing polypeptides and polynucleotides into plant cells include microinjection (Crossway et al. (1986) Biotechniques 4:320-334), electroporation (Riggs et al. (1986) Proc. Natl. Acad. Sci. USA 83:5602-5606, Agrobacterium-mediated transformation (U.S. Pat. No. 5,563,055 and U.S. Pat. No. 5,981,840), direct gene transfer (Paszkowski et al. (1984) EMBO J. 3:2717-2722), and ballistic particle acceleration (see, for example, U.S. Pat. No. 4,945,050; U.S. Pat. No. 5,879,918; U.S. Pat. Nos. 5,886,244; and, 5,932,782; Tomes et al. (1995) in Plant Cell, Tissue, and Organ Culture: Fundamental Methods, ed. Gamborg and Phillips (Springer-Verlag, Berlin); McCabe et al. (1988) Biotechnology 6:923-926); and Lec1 transformation (WO 00/28058). Also see Weissinger et al. (1988) Ann. Rev. Genet. 22:421-477; Sanford et al. (1987) Particulate Science and Technology 5:27-37 (onion); Christou et al. (1988) Plant Physiol. 87:671-674 (soybean); McCabe et al. (1988) Bio/Technology 6:923-926 (soybean); Finer and McMullen (1991) In Vitro Cell Dev. Biol. 27P: 175-182 (soybean); Singh et al. (1998) Theor. Appl. Genet. 96:319-324 (soybean); Datta et al. (1990) Biotechnology 8:736-740 (rice); Klein et al. (1988) Proc. Natl. Acad. Sci. USA 85:4305-4309 (maize); Klein et al. (1988) Biotechnology 6:559-563 (maize); U.S. Pat. Nos. 5,240,855; 5,322,783; and, 5,324,646; Klein et al. (1988) Plant Physiol. 91:440-444 (maize); Fromm et al. (1990) Biotechnology 8:833-839 (maize); Hooykaas-Van Slogteren et al. (1984) Nature (London) 311:763-764; U.S. Pat. No. 5,736,369 (cereals); Bytebier et al. (1987) Proc. Natl. Acad. Sci. USA 84:5345-5349 (Liliaceae); De Wet et al. (1985) in The Experimental Manipulation of Ovule Tissues, ed. Chapman et al. (Longman, New York), pp. 197-209 (pollen); Kaeppler et al. (1990) Plant Cell Reports 9:415-418 and Kaeppler et al. (1992) Theor. Appl. Genet. 84:560-566 (whisker-mediated transformation); D'Halluin et al. (1992) Plant Cell 4:1495-1505 (electroporation); Li et al. (1993) Plant Cell Reports 12:250-255 and Christou and Ford (1995) Annals of Botany 75:407-413 (rice); Osjoda et al. (1996) Nature Biotechnology 14:745-750 (maize via Agrobacterium tumefaciens); all of which are herein incorporated by reference.

In specific embodiments, the polynucleotide of the invention can be provided to one or more plant cells using a variety of transient transformation methods. Such transient transformation methods include, but are not limited to, the introduction of a polypeptide of interest directly into a cell of an embryo sac or portion thereof or the introduction of a transcript encoding the polypeptide of interest into an embryo sac or portion thereof. Such methods include, for example, microinjection or particle bombardment. See, for example, Crossway et al. (1986) Mol Gen. Genet. 202:179-185; Nomura et al. (1986) Plant Sci. 44:53-58; Hepler et al. (1994) Proc. Natl. Acad. Sci. 91: 2176-2180 and Hush et al. (1994) The Journal of Cell Science 107:775-784, all of which are herein incorporated by reference. Alternatively, a polynucleotide of interest can be transiently transformed into the plant using techniques known in the art. Such techniques include viral vector system and the precipitation of the polynucleotide in a manner that precludes subsequent release of the DNA. Thus, the transcription from the particle-bound DNA can occur, but the frequency with which its released to become integrated into the genome is greatly reduced. Such methods include the use particles coated with polyethylimine (PEI; Sigma #P3143).

The developing endosperm or embryo in the fertilized cultured embryo sacs can be targeted for transformation, for example by microinjection, in order to, for example, to evaluate the strength of regulatory sequences, such as a specific promoter, in that tissue. For example, endosperm cells are transformed with a foreign gene and allowed to develop in vitro. The foreign DNA, for example, may be a reporter gene such as ZsYellow or GFP operably linked to a promoter to be tested. ZsYellow or GFP expression is assayed or quantified by methods well known to the skilled artisan such as, for example, detecting fluorescence. Based on the level of expression of the foreign gene in the endosperm or embryo a prediction can be made as to the whether the selected regulatory sequence will drive sufficient expression of the foreign gene to modify the phenotype of the seed.

Methods for introducing a polynucleotide of interest into cereal embryo sacs, particularly maize embryo sacs, have been previously reported. U.S. Pat. No. 6,300,543 describes a method for the microinjection of foreign DNA into zygotes in fertilized maize embryo sacs and the regeneration of transformed plants therefrom.

In other embodiments, the polynucleotide of interest may be introduced into plant cells by contacting plant cells with a virus or viral nucleic acids. Generally, such methods involve incorporating a nucleotide construct of the invention within a viral DNA or RNA molecule. It is recognized that the a polypeptide of interest may be initially synthesized as part of a viral polyprotein, which later may be processed by proteolysis in vivo or in vitro to produce the desired recombinant protein. Further, it is recognized that promoters of the invention also encompass promoters utilized for transcription by viral RNA polymerases. Methods for introducing polynucleotides into plant cells and expressing a protein encoded therein, involving viral DNA or RNA molecules, are known in the art. See, for example, U.S. Pat. Nos. 5,889,191, 5,889,190, 5,866,785, 5,589,367, 5,316,931, and Porta et al. (1996) Molecular Biotechnology 5:209-221; herein incorporated by reference.

Methods are known in the art for the targeted insertion of a polynucleotide at a specific location in the plant genome. In one embodiment, the insertion of the polynucleotide at a desired genomic location is achieved using a site-specific recombination system. See, for example, WO 99/25821, WO 99/25854, WO 99/25840, WO 99/25855, and WO 99/25853, all of which are herein incorporated by reference. Briefly, the polynucleotide of the invention can be contained in transfer cassette flanked by two non-recombinogenic recombination sites. The transfer cassette is introduced into a plant having stably incorporated into its genome a target site which is flanked by two non-recombinogenic recombination sites that correspond to the sites of the transfer cassette. An appropriate recombinase is provided and the transfer cassette is integrated at the target site. The polynucleotide of interest is thereby integrated at a specific chromosomal position in the plant genome.

As used herein, the term plant includes plant cells, plant protoplasts, plant cell tissue cultures from which maize plant can be regenerated, plant calli, plant clumps, and plant cells that are intact in plants or parts of plants such as embryos, embryo sacs, pollen, ovules, endosperm, seeds, leaves, flowers, branches, fruit, kernels, ears, cobs, husks, stalks, roots, root tips, anthers, and the like. Grain is intended to mean the mature seed produced by commercial growers for purposes other than growing or reproducing the species. Progeny, variants, and mutants of the regenerated plants are also included within the scope of the invention, provided that these parts comprise the introduced polynucleotides.

The phrase “embryo sac or portion therein” is used throughout this disclosure. Unless otherwise indicated, “portion thereof” in this context refers to any part of an embryo sac that is less than a whole embryo sac. Such a “portion” can be, for example a single endosperm or embryo cell, an embryo sac with the embryo removed, an embryo, endosperm, aleurone, and the like.

The methods of the present invention are directed to plants, particularly cereal plants and cells, tissues, and seeds thereof. Such cereal plants include, but are not limited to, maize or corn (Zea mays), wheat (Triticum aestivum, Triticum turgidum subsp. durum), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana) triticale (×Triticosecale), oats (Avena sativa), barley (Hordeum vulgare), teff (Eragrostis tef), and spelt (Triticum spelta).

In specific embodiments, a polypeptide or the polynucleotide of interest is introduced into at least one plant cell. Subsequently, a plant cell having the introduced sequence of the invention can be selected using methods known to those of skill in the art such as, but not limited to, Southern blot analysis, DNA sequencing, PCR analysis, or phenotypic analysis. A plant cell or plant part altered or modified by the foregoing embodiments is exposed to proliferation medium for a time sufficient to modulate the concentration and/or activity of polypeptides of the present invention in the plant cell or part.

Thus, the present invention provides methods for modulating the level of a polypeptide of interest in a cereal embryo sac or portion thereof. These methods involve increasing or decreasing the level of a polypeptide of interest in a plant or cell thereof. The polypeptide of interest can be encoded by an endogenous gene of said embryo sac or by a transgene. In an embodiment of the invention, the transgene is a reporter or marker gene such as, for example, ZsYellow or green fluorescent protein (GFP).

The methods for modulating the level of a polypeptide of interest in a plant or cell thereof comprise introducing into a cultured cereal embryo sac or portion thereof a polynucleotide of interest, wherein the embryo sac or portion thereof is cultured by the methods of the present invention. The portion of the embryo sac can be, for example, the endosperm, starchy endosperm, aleurone, the embryo, a transfer cell, and embryo surrounding region. The polynucleotide of interest can be, but need not be, stably integrated into the genome of at least one cell of said embryo sac or said portion. In certain embodiments of the invention, the polynucleotide of interest is operably linked to a promoter that drives expression in said embryo sac or portion thereof, particularly the endosperm, the embryo, or both the endosperm and the embryo. In one embodiment of the invention, the polynucleotide of the polynucleotide of interest comprises a hairpin construct suitable for decreasing the level of said polypeptide of polypeptide of interest by RNAi. In another embodiment, the polynucleotide of interest is encoded by a transgene that is introduced into said embryo sac or portion thereof before, at the same time as, or after the polynucleotide of interest is introduced into said embryo sac or portion thereof. Additionally provided are transformed plant tissues and plant cells that comprise stably integrated in their genomes the polynucleotide of interest.

It is also recognized that the level and/or activity of the polypeptide may be modulated by employing a polynucleotide that is not capable of directing, in a transformed plant cell, the expression of a protein or an RNA. For example, the polynucleotides of the invention may be used to design polynucleotide constructs that can be employed in methods for altering or mutating a genomic nucleotide sequence in an organism. Such polynucleotide constructs include, but are not limited to, RNA:DNA vectors, RNA:DNA mutational vectors, RNA:DNA repair vectors, mixed-duplex oligonucleotides, self-complementary RNA:DNA oligonucleotides, and recombinogenic oligonucleobases. Such nucleotide constructs and methods of use are known in the art. See, U.S. Pat. Nos. 5,565,350; 5,731,181; 5,756,325; 5,760,012; 5,795,972; and 5,871,984; all of which are herein incorporated by reference. See also, WO 98/49350, WO 99/07865, WO 99/25821, and Beetham et al. (1999) Proc. Natl. Acad. Sci. USA 96:8774-8778; herein incorporated by reference.

It is therefore recognized that methods of the present invention do not depend on the incorporation of the entire polynucleotide into the genome, only that the plant or cell thereof is altered as a result of the introduction of the polynucleotide into a cell. In one embodiment of the invention, the genome may be altered following the introduction of the polynucleotide into a cell. For example, the polynucleotide, or any part thereof, may incorporate into the genome of the plant. Alterations to the genome of the present invention include, but are not limited to, additions, deletions, and substitutions of nucleotides into the genome. While the methods of the present invention do not depend on additions, deletions, and substitutions of any particular number of nucleotides, it is recognized that such additions, deletions, or substitutions comprises at least one nucleotide.

In one embodiment, the activity and/or level of the polypeptide of interest is increased. An increase in the level and/or activity of the polypeptide of interest can be achieved by providing to the plant the polypeptide of interest. As discussed elsewhere herein, many methods are known the art for providing a polypeptide to a plant including, but not limited to, direct introduction of the polypeptide into the plant, introducing into the plant (transiently or stably) a polynucleotide construct encoding the polypeptide of interest. It is also recognized that the methods of the invention may employ a polynucleotide that is not capable of directing, in the transformed plant, the expression of a protein or an RNA. Thus, the level and/or activity of a polypeptide of interest may be increased by altering the gene encoding the polypeptide or its promoter. See, e.g., Kmiec, U.S. Pat. No. 5,565,350; Zarling et al., PCT/US93/03868.

In other embodiments, the activity and/or level of the polypeptide of interest is reduced or eliminated by introducing into a plant a polynucleotide that inhibits the level or activity of the the polypeptide of interest. The polynucleotide may inhibit the expression of the polypeptide of interest directly, by preventing translation of the messenger RNA encoding the polypeptide, or indirectly, by encoding a polypeptide that inhibits the transcription or translation of a gene encoding the polypeptide of interest. Methods for inhibiting or eliminating the expression of a gene in a plant are well known in the art, and any such method may be used in the present invention to inhibit the expression of any particular gene or genes in a plant. In other embodiments of the invention, the activity of the polypeptide of interest is reduced or eliminated by transforming a plant cell with a sequence encoding a polypeptide that inhibits the activity of the polypeptide. In other embodiments, the activity of a polypeptide of interest may be reduced or eliminated by disrupting the gene encoding the polypeptide of interest.

Reduction of the activity of specific genes (also known as gene silencing or gene suppression) is desirable for several aspects of genetic engineering in plants. Many techniques for gene silencing are well known to one of skill in the art, including, but not limited to, antisense technology (see, e.g., Sheehy et al. (1988) Proc. Natl. Acad. Sci. USA 85:8805-8809; and U.S. Pat. Nos. 5,107,065; 5,453,566; and 5,759,829); cosuppression (e.g., Taylor (1997) Plant Cell 9:1245; Jorgensen (1990) Trends Biotech. 8(12):340-344; Flavell (1994) Proc. Natl. Acad. Sci. USA 91:3490-3496; Finnegan et al. (1994) Bio/Technology 12:883-888; and Neuhuber et al. (1994) Mol. Gen. Genet. 244:230-241); RNA interference (Napoli et al. (1990) Plant Cell 2:279-289; U.S. Pat. No. 5,034,323; Sharp (1999) Genes Dev. 13:139-141; Zamore et al. (2000) Cell 101:25-33; and Montgomery et al. (1998) Proc. Natl. Acad. Sci. USA 95:15502-15507), virus-induced gene silencing (Burton et al. (2000) Plant Cell 12:691-705; and Baulcombe (1999) Curr. Op. Plant Bio. 2:109-113); target-RNA-specific ribozymes (Haseloff et al. (1988) Nature 334: 585-591); hairpin structures (Smith et al. (2000) Nature 407:319-320; WO 99/53050; WO 02/00904; WO 98/53083; Chuang and Meyerowitz (2000) Proc. Natl. Acad. Sci. USA 97:4985-4990; Stoutjesdijk et al. (2002) Plant Physiol. 129:1723-1731; Waterhouse and Helliwell (2003) Nat. Rev. Genet. 4:29-38; Pandolfini et al. BMC Biotechnology 3:7, U.S. Patent Publication No. 20030175965; Panstruga et al. (2003) Mol. Biol. Rep. 30:135-140; Wesley et al. (2001) Plant J. 27:581-590; Wang and Waterhouse (2001) Curr. Opin. Plant Biol. 5:146-150; U.S. Patent Publication No. 20030180945; and, WO 02/00904, all of which are herein incorporated by reference); ribozymes (Steinecke et al. (1992) EMBO J. 11:1525; and Perriman et al. (1993) Antisense Res. Dev. 3:253); oligonucleotide-mediated targeted modification (e.g., WO 03/076574 and WO 99/25853); Zn-finger targeted molecules (e.g., WO 01/52620; WO 03/048345; and WO 00/42219); transposon tagging (Maes et al. (1999) Trends Plant Sci. 4:90-96; Dharmapuri and Sonti (1999) FEMS Microbiol. Lett. 179:53-59; Meissner et al. (2000) Plant J. 22:265-274; Phogat et al. (2000) J. Biosci. 25:57-63; Walbot (2000) Curr. Opin. Plant Biol. 2:103-107; Gai et al. (2000) Nucleic Acids Res. 28:94-96; Fitzmaurice et al. (1999) Genetics 153:1919-1928; Bensen et al. (1995) Plant Cell 7:75-84; Mena et al. (1996) Science 274:1537-1540; and U.S. Pat. No. 5,962,764); each of which is herein incorporated by reference; and other methods or combinations of the above methods known to those of skill in the art.

It is recognized that with the polynucleotides of the invention, antisense constructions, complementary to at least a portion of the messenger RNA (mRNA) encoding the polypeptide of interest can be constructed. Antisense nucleotides are constructed to hybridize with the corresponding mRNA. Modifications of the antisense sequences may be made as long as the sequences hybridize to and interfere with expression of the corresponding mRNA. In this manner, antisense constructions having 70%, optimally 80%, more optimally 85% sequence identity to the corresponding antisensed sequences may be used. Furthermore, portions of the antisense nucleotides may be used to disrupt the expression of the target gene. Generally, sequences of at least 20 nucleotides, 50 nucleotides, 100 nucleotides, 200 nucleotides, 300, 400, 450, 500, 550, or greater may be used.

The methods of the present invention can also be used to with a polynucleotide of interest in the sense orientation to suppress the expression of endogenous genes in plants. Methods for suppressing gene expression in plants using polynucleotides in the sense orientation are known in the art. The methods generally involve transforming plant cells with a DNA construct comprising a promoter that drives expression in a plant operably linked to at least a portion of a polynucleotide that corresponds to the transcript of the endogenous gene. Typically, such a nucleotide sequence has substantial sequence identity to the sequence of the transcript of the endogenous gene, optimally greater than about 65% sequence identity, more optimally greater than about 85% sequence identity, most optimally greater than about 95% sequence identity. See, U.S. Pat. Nos. 5,283,184 and 5,034,323; herein incorporated by reference. Thus, the methods of the present invention can be used to reduce or eliminate the activity of a polypeptide of interest. More than one method may be used to reduce the activity of a single polypeptide of interest. In addition, combinations of methods may be employed to reduce or eliminate the activity of one or more polypeptides of interest.

The embryo sacs or portions thereof of the present invention find further use in phage display library screening. For example, in vitro cultured endosperm can be used to pan a phage display library containing proteins from a variety of sources to identify proteins or peptides that interacting with proteins on the surface of endosperm cells.

The methods of the present invention find further use in assessing the zygotic component of grain yield. In vitro cultured endosperm of the present invention can be used to asses the contribution of the zygotic genome on grain yield that is normally confounded with maternal effect when studied in intact plants

The methods of the invention can be used to introduce a polynucleotide of interest that is operable linked to a promoter nucleotide sequence into a host plant cell in order to vary the phenotype of a plant cell, plant tissue, or plant. Of particular interest, are genes that impact grain characteristics, particularly endosperm characteristics.

Genes of interest are reflective of the commercial markets and interests of those involved in the development of the cereal crop, particularly the cereal grain. Crops and markets of interest change, and as developing nations open up world markets, new crops and technologies will emerge also. In addition, as our understanding of agronomic traits and characteristics such as yield and heterosis increase, the choice of genes for transformation will change accordingly. The quality of grain is reflected in traits such as levels and types of oils, saturated and unsaturated, quality and quantity of essential amino acids, the levels of cellulose, the levels and types of starch and other polysaccharides. In corn, for example, modified hordothionin proteins are described in U.S. Pat. Nos. 5,703,049, 5,885,801, 5,885,802, and 5,990,389.

Commercial traits can also be encoded on a gene or genes that could increase for example, starch for ethanol production, or provide expression of proteins. Another important commercial use of transformed plants is the production of polymers and bioplastics such as described in U.S. Pat. No. 5,602,321. Genes such as β-ketothiolase, PHBase (polyhydroxyburyrate synthase), and acetoacetyl-CoA reductase (see Schubert et al. (1988) J. Bacteriol. 170:5837-5847) facilitate expression of polyhyroxyalkanoates (PHAs).

Exogenous products include plant enzymes and products as well as those from other sources including prokaryotes and other eukaryotes. Such products include enzymes, cofactors, hormones, and the like. The level of proteins, particularly modified proteins having improved amino acid distribution to improve the nutrient value of the plant, can be increased. This is achieved by the expression of such proteins having enhanced amino acid content.

The present invention further provides methods for determining the effect of a chemical of interest on endosperm development: Such methods make use of endosperm cells that are cultured by the methods disclosed herein. The methods for determining the effect of a chemical of interest on endosperm development comprise contacting an embryo sac or portion thereof with a chemical of interest, wherein said embryo sac or portion thereof is cultured by the methods of the present invention. The embryo sac or portion thereof can be contacted with the chemical of interest before, during, and/or after exposure to a proliferation medium of the invention. The methods further involve monitoring the development of said endosperm in said embryo sac or portion thereof. In certain embodiments, the methods comprise the use of embryo sacs or portions thereof that have been transformed with a marker gene, particularly a marker gene that encodes a fluorescent protein including, but not limited to, ZsYellow and GFP. In such embodiments, the marker gene is operably linked to a promoter that drives expression in the endosperm or part thereof, such as, for example, the Ltp2 promoter or gamma-zein promoter.

The methods of the present invention find further use in determining the subcellular localization of a protein in cells produced by the in vitro culture methods disclosed herein. In one embodiment, the invention provides methods for determining the subcellular localization of a protein in endosperm cells. Such methods make use of endosperm cells that cultured by the methods disclosed herein. The methods for determining the subcellular localization of a protein in endosperm cells involve introducing into an endosperm cell a nucleic acid construct comprising a polynucleotide encoding a protein of interest. Such a polynucleotide is operably linked to a promoter that drives expression in an endosperm cell. The polynucleotide is also operably linked to a marker gene encoding a fluorescent protein so as to allow for the production of a fusion protein comprising said protein of interest and said fluorescent protein in a transformed embryo sac cell, particularly in an endosperm cell therein. The methods further involve determining the subcellular localization of the protein in the endosperm by detecting the subcellular location of fluorescence from said fluorescent protein by, for example, microscopy, and particularly confocal microscopy. Accordingly, the methods of the present invention find use in determining the subcellular localization of a protein in any one or more cellular compartment, structure, and/or organelle including, but not limited to, the plasmalemma, the cell wall, mitochondria, amyloplasts, the vacuole, peroxisomes, the endoplasmic reticulum, golgi bodies, the nucleus, and the cytoplasm.

The present invention provides an in vitro endosperm culture system that can be used to study the controls of endosperm development without the confounding effects of maternal signaling. Accordingly, the in vitro endosperm system of the present invention finds further use in studying separately the contribution of the sink and the source components of grain yield.

### EXAMPLE 1

### Establishment of the Endosperm In Vitro Culture System (EICS)

Fertilized maize (Zea mays L.) embryo sacs from ovules harvested shortly after fertilization were dissected out with little or no adhering maternal (nucellus) tissue using forceps from intact ovules and transferred to culture medium for growth. Ovules harvested at 6 DAP (days after pollination) were routinely used to establish the cultures. However, earlier stage embryo sacs also develop and grow, such as, for example, 3 DAP embryo sacs. Generally, 3 DAP fertilized embryo sacs are at the earliest developmental stage in which embryo sacs can be dissected substantially free from the surrounding nucellus tissue. Typically, it is not possible to mechanically isolate fertilized embryo sacs at 1 DAP and 2 DAP that lack significant amounts of adhereing nucellus tissue.

The 6 DAP embryo sacs that are routinely used in the methods of the present invention grow and develop into an endosperm when cultured by the methods disclosed herein that is similar to the endosperm that is produced from the fertilized embryo sacs isolated at 1 DAP grows in the nucellus slab culture system (Laurie et al. (1999) In Vitro Cell Dev. Biol.—Plant 35:320-325). The cultured fertilized embryo sacs of the present invention, unlike nucellus slab culture embryo sacs, are not surrounded by tissue from the ovary wall and nucellus, both of which continue to grow in nucellus slab culture system further obscuring the embryo sac and endosperm therein and thus, limiting extended studies of in vitro endosperm development. In addition to the use of embryo sacs isolated from 3 to 6 DAP, EICS can also be established from later stages. Embryo sacs isolated at 10, 11, and 12 DAP have also been used to establish EICS.

Embryo sacs were isolated from maize ovules at 6 DAP substantially free from maternal tissues, with or without the embryo, and placed either on solid medium or in liquid medium. Using embryo sacs from lines that comprise transgenes that drive the expression of fluorescent protein genes in the aleurone cells (ZsYellow), starchy endosperm cells (amCFP), and transfer cells cells (dsRFP), the progression of development of the in vitro endosperm was observed to be similar to that of the in planta endosperm. Similar to in planta endosperm, the surface layer of the in vitro cultured endosperm consisted of aleurone cells with an interior mass of starchy endosperm cells. Occasionally, it was observed that cells positioned adjacent to interior voids sometimes develop into aleurone cells in the interior of the in vitro endosperm. In general, similar to in planta endosperm, aleurone cells develop on all surfaces of the endosperm including the surface lining the voids, even if the cells on the surface of the voids, prior to the development of the void, had differentiated into starchy endosperm cells. The development of aleurone cells on all surfaces of the endosperm is consistent with the “surface” rule that was proposed by Olsen ((2004) Maydica 49: 37-40) following the observation of spherical bodies of endosperm in in planta grown endosperm of defective maize kernel mutants.

At the time of initiation of the in vitro endosperm cultures at 6 DAP, the isolated fertilized embryo sac is pointed and appears translucent. Soon after, at 2 days in in vitro culture (DIV), the endosperm appears opaque, and two days later the shape becomes more rounded. Similar to the dissected in planta endosperm, the surface of the in vitro endosperm stays relatively smooth up to 10 DIV. The overall expansion of the in vitro endosperm is small compared to in planta grown endosperm, which we assumed is caused by a deficiency of influx of carbon into the in vitro endosperm compared to in planta with its highly effective placento-chalazal transfer cell complex for solute transfer from the source to the sink tissues. In in vitro cultures that are initiated without removing the embryo, the embryos develop and precociously germinate around 20-25 days in in vitro culture (DIV). We do not observe obvious differences in the morphology of in vitro endosperm grown with or without the embryo attached. After the first wave of mitotic divisions, ending approximately after 10 days in culture, the surface cell layer(s) continue to undergo active mitotic divisions leading to “bulges” that represent “mini-endosperms” consisting of an exterior cell surface of aleurone cells and an interior mass of starchy endosperm cells.

Various genotypes of maize have been used successfully to initiate EICS. All of the genotypes tested have responded favorably, including B73, GS3, HG11, Gaspee Flint, dek1/+ heterozygotes, sal1 plants, untransformed plants, as well as transgenic lines in different backgrounds harboring various constructs.

The composition of the EICS culture medium, also referred to herein as proliferation medium, has been previously described by Laurie et al. (1999) In Vitro Cell Dev. Biol.—Plant 35:320-325. In addition to solid proliferation medium (i.e., gelling agent added), liquid proliferation medium can also be used. The liquid medium contains the same components as the solid medium except the liquid medium lacks the gelling agent. Typically, 1 L of solid proliferation medium comprises the following components: 950 mL of polished D-1H₂O, 4.3 g MS salts (Gibco No. 11117) (yields a 1× concentration in a final volume of 1 L), 5 mL MS Vitamins Stock Solution (Gibco No. 36J) (yields a 1× concentration in a final volume of 1 L), 1.25 mL of a 4 mg/mL solution of thiamine HCL, 0.1 mL of a 1.0 mg/mL solution of 6-benzylaminopurine (BAP), 150 g sucrose, and 3 g Gelrite. The pH is adjusted to 5.8 with KOH before sterilization by autoclaving.

### EXAMPLE 2

### Characterization of EICS Growth and Development

To monitor growth and development, EICS was initiated from plants that are transgenic for the Ltp2::ZsYellow construct. The Ltp2 promoter from barley drives transcription specifically in the aleurone layer, and thus represents a convenient tissue-specific marker to monitor aleurone cell differentiation (Kalla et al. (1994) Plant J. 6: 849-860). Starchy endosperm cells can be identified on the basis of their starch content. After transfer of the developing embryo sacs to the culture medium, growth of the endosperm continues. After four days in culture, aleurone cells fluoresce strongly from the Ltp2::ZsYellow construct demonstrating that the onset of the fluorescent marker expression in EICS corresponds closely to that of in planta grown endosperms.

To monitor the growth pattern of EICS endosperms, transverse sections of endosperm expressing the Ltp2::ZsYellow were studied. In these sections, the presence of the fluorescent marker clearly demonstrate that the cells of the surface aleurone cell layer continue to divide and maintain their differentiation status as aleurone cells, and that the underlying cells continue to divide and accumulate starch granules indicative of their differentiation status as starchy endosperm cells. Functional transfer cells are not identifiable in EICS endosperms as based on cell morphology in the region where transfer cells develop in planta. Embryo surrounding cells are present based on the morphology and location of these cells.

### EXAMPLE 3

### Characterization of EICS Growth and Development with Endosperm Developmental Mutants

To investigate whether the EICS endosperm accurately reflects the phenotype of endosperm of known endosperm developmental mutants, the defective kernel1 (dek1) endosperm, lacking aleurone cells (Becraft et al. (1996) Science 273: 1406-1409; Lid et al. (2004) Planta 218: 370-378) and superal1 (sal1-2) (Shen et al. (2003) Proc. Natl. Acad. Sci. USA 100: 6552-6557) endosperm with two layers of aleurone cells were cultured. Similar to in planta endosperms, dek1 endosperms lack a surface layer of aleurone cells and have large starchy endosperm cells occupying the surface position. Also, EICS of homozygous sal1-2 kernels develop two layers of aleurone cells similar to in planta grown endosperm. Thus, in vitro cultured endosperm of known endosperm developmental mutants that is produced by the methods disclosed herein displays a level of endosperm organization that is similar to the organization of such endosperm mutants in planta. Therefore, the methods disclosed herein find use with both wild-type and endosperm developmental mutants.

### EXAMPLE 4

### Transformation of EICS Cells

To investigate whether the EICS endosperm cells are amenable to commonly practiced methods of gene transformation, cultured wild-type endosperm were bombarded with the two different Ltp2::ZsYellow constructs. After continued culture overnight, a high proportion of the aleurone cells were expressing the fluorescent marker. Furthermore, fluorescent positive cells divided and formed sectors after continued cultivation. Thus, these results demonstrate that EICS is amenable to commonly practiced gene transformation methods and that EICS can be used to directly assess the overexpression of introduced genes and/or the inhibiting the expression of endogenous genes by methods such as, for example, antisense suppression and RNA interference (RNAi).

### EXAMPLE 5

### RNAi Downregulation of Gene Expression in In Vitro Cultured Endosperm

Maize embryo sacs were harvested at 6 DAP and grown for 3-5 days on solid proliferation medium before bombardment. Two RNAi constructs were used. The first construct (hairpin construct 1) comprises the first 293 bp of the ZsYellow1 (BD Biosciences Clontech, Palo Alto, Calif., USA) coding sequence inserted in reverse orientation behind the operably linked aleurone-preferred promoter, Ltp2. This fragment is then followed by a spliceable Adh1 intron from maize and the complete coding sequence for ZsYellow1 in the sense orientation. The second construct (hairpin construct 2) comprises the same inverted repeat of ZsYellow1 as described for construct 1 above, with a constitutive ubiquitin promoter/intron replacing the aleurone-preferred promoter.

Prior to bombardment the embryo sacs were arranged in the center of the plate of solid proliferation medium. The constructs were treated as follows prior to bombardment:

Preparation of DNA for 10 bombardments:
1. Add each of the following sequentially: 100 μL prepared 0.6μ gold particles (1.5 mg particles) in water in a siliconized tube; 10 μL plasmid DNA (0.1 to 1 μg/μL); 100 μL 2.5 M CaCl₂, 10 μL 0.1 M spermidine.
2. Vortex at 3-4 setting for 10 minutes.
3. Spin and remove the liquid from the tube.
4. Wash with 500 μL 100% ethanol.
5. Spin and remove liquid from tube.
6. Add 105 μL of 100% ethanol for 10 bombardments (10 μL/bombardment).
7. Briefly sonicate before use.

Particle bombardment was conducted at 1100 psi.

Three different combinations of constructs were co-bombarded. All of the vector solutions were at a concentration of 125 ng DNA/μL. In the first combination, 5 μL of the vector comprising Ltp2::ZsYellow and 5 μL of the vector comprising hairpin construct 1 were co-bombarded. In the first combination, 5 μL of the vector comprising Ltp2::ZsYellow and 5 uL of the vector comprising hairpin construct 2 were co-bombarded. In the third combination, which served as the control, 5 μL of the vector comprising Ltp2::ZsYellow and 5 uL of the vector alone (i.e., no ZsYellow insert) were co-bombarded.

When the embryo sacs were observed at 24 hours after bombardment, a 90% reduction in the fluorescence from the Ltp::ZsYellow marker was observed in the embryo sacs bombarded with either of the two hairpin constructs relative to the control embryo sacs bombarded the third combination (i.e., no hairpin construct). Thus, the results demonstrate that the in vitro cultured endosperm of the present invention finds use in methods for downregulating gene expression by RNA

### EXAMPLE 6

### Analysis of Molecular Markers for Starchy Endosperm Cells, Aleurone Cells, and Transfer Cells in Cultured Endosperm

In order to monitor differentiation and growth of in vitro grown endosperm with cellular resolution, we developed a transgenic maize line expressing the cyan fluorescent protein (AmCFP1) in starchy endosperm cells under the control of the maize 27 kDa γ-zein promoter (Ueda et al. (1991) Theor. Appl. Genet. 82:93-100); Russell et al. (1997) Transgenic Res. 6:157-168), the yellow fluorescence protein (ZsYellow1) in aleurone cells driven by the barley Ltp2 promoter (Kalla et al. (1994) Plant J. 6:849-860 and the red fluorescent protein (DsRed2) in transfer cells directed by the maize End1 promoter (unpublished). We refer to this line as the “triple line”. In addition, we used Massive Parallel Signature Sequencing (MPSS) (Brenner (2000), Nature Biotechnology 18:630-634) to monitor steady state levels of the triple line marker genes 27 kDa γ-zein, End1 and the maize homologue of the barley Ltp2 gene, zmLtp2 (Table 1). Tissue sources for the MPSS experiments included isolated intact endosperm at 12 and 18 DAP, isolated starchy endosperm cells from 18 and 27 DAP, and isolated basal endosperm at 12 and 27 DAP (Table 1). As expected from published data (Ueda et al. (1991) Theor. Appl. Genet. 82:93-100); Russell et al. (1997) Transgenic Res. 6:157-168), the 27 kDa γ-zein transcript is expressed at high levels in in planta endosperm at 12 DAP, increasing at 18 DAP, reaching almost half a million per million transcripts (tpm) in dissected starchy endosperm at 18 DAP. In addition to starchy endosperm, 27 kDa γ-zein transcripts are also present in dissected aleurone layers. Most likely, this transcript represents contaminating starchy endosperm cells in this preparation.

TABLE 1
Triple Line Marker Gene Expression in In Planta and In Vitro
Endosperm (values expressed as transcripts per million (tpm))

| | | Marker gene: | | | | | | | | |
|---|---|---|---|---|---|---|---|---|---|---|
| | | 27 kDa γ-zein | | | End1 | | | ZmLtp2 | | |
| Stage (DAP): | | 12 | 18 | 27 | 12 | 18 | 27 | 12 | 18 | 27 |
| In | Total | 100733 | 219844 | | 11092 | 295 | | 321 | 0 | |
| planta | endosperm | | | | | | | | | |
| Tissue | Dissected | | 80665 | 11259 | | 1619 | 329 | | 4917 | 20347 |
| source | aleurone | | | | | | | | | |
| | Dissected | | 485637 | 162625 | | 0 | 0 | | 0 | 0 |
| | starchy | | | | | | | | | |
| | endosperm | | | | | | | | | |
| | Basal transfer | 10758 | | 61738 | 6395 | | 540 | 150 | | 3602 |
| | cell layer | | | | | | | | | |

| | | | | | | |
|---|---|---|---|---|---|---|
| Stage (DIV): | 6 | 15 | 6 | 15 | 6 | 15 |
| In vitro endosperm | 146725 | 40602 | 3254 | 5179 | 1308 | 117 |

TABLE 2
Expression of Aleurone Specific or Preferred Transcripts in
In Planta and In Vitro Endosperm (values expressed as tpm)

| | | Marker gene: | | | | | | | | |
|---|---|---|---|---|---|---|---|---|---|---|
| | | | | | | | | THZ2_MAIZE | | |
| | | | | | | | | GAMMA- | | |
| | | NLTP_MAIZE | | | Globulin 1 | | | ZEATHIONIN 2 | | |
| Stage (DAP): | | 12 | 18 | 27 | 12 | 18 | 27 | 12 | 18 | 27 |
| In | Total | 413 | 5944 | | 197 | 0 | | 181 | 3592 | |
| planta | endosperm | | | | | | | | | |
| Tissue | Dissected | | 74646 | 104703 | | 906 | 8820 | | 27213 | 52027 |
| source | aleurone | | | | | | | | | |
| | Dissected | | 897 | 32 | | 0 | 0 | | 39 | 20 |
| | starchy | | | | | | | | | |
| | endosperm | | | | | | | | | |
| | Basal | 207 | | 3159 | 0 | | 643 | 30 | | 1698 |
| | transfer cell | | | | | | | | | |
| | layer | | | | | | | | | |

| | | | | | | |
|---|---|---|---|---|---|---|
| Stage (DIV): | 6 | 15 | 6 | 15 | 6 | 15 |

In planta, the AmCFP1 marker first appears in starchy endosperm at 12 DAP, the fluorescence signal increasing in intensity towards 20 DAP (data not shown), according well with the level of 27 kDa γ-zein transcript seen in the MPSS analysis. Similarly, the AmCFP1 fluorescence marker becomes visible in in vitro cultured endosperm at 6 DIV (12 DAP). The activity of the 27 kDa γ-zein promoter as detected by the cyan fluorescence marker in vitro was also confirmed by the MPSS transcript profiling data from in vitro grown endosperm, where high levels of the 27 kDa γ-zein transcript were detected at 6 DIV (Table 1). In contrast to in planta, however, where the 27 kDa γ-zein transcript level increases towards later developmental stages, this mRNA decreased more than three fold between 6 and 15 DIV in in vitro grown endosperm.

In order to determine if the decreased level of the 27 kDa γ-zein transcript was representative of storage protein transcripts of in vitro cultured endosperm, we also examined the level of the 16 kD γ-zein, 19 kDa alpha-zein_B3 and 50 kDa γ-zein transcripts (Woo et al. (2001) Plant Cell 13:2297-2317) (Table 3). Comparing 6 DIV in vitro endosperm with 12 DAP in planta endosperm, the only significant difference detectable was for the 16 kDa γ-zein, which was almost three-fold higher in the in vitro endosperm. However, comparing 15 DIV in vitro grown endosperm with 18 DAP in planta endosperm, all storage protein transcript levels are considerably higher in planta than in vitro. Also, storage protein transcript levels of in vitro cultured endosperm were reduced at 15 DIV compared to 6 DIV (Table 3). In contrast, aleurone preferred transcripts of in vitro cultured endosperm increased between 6 and 15 DIV (Table 2).

TABLE 3
Expression of Starchy Endosperm Cell Specific or Preferred Transcripts
in In Planta and In Vitro Endosperm (values expressed as tpm)

| | | Marker gene | | | | | | | | |
|---|---|---|---|---|---|---|---|---|---|---|
| | | 16 kDa γ-zein | | | 19 kDa α-zein_B3 | | | 50 kDa γ-zein | | |
| Stage (DAP) | | 12 | 18 | 27 | 12 | 18 | 27 | 12 | 18 | 27 |
| In | Total | 72543 | 273754 | | 26154 | 305761 | | 1704 | 25413 | |
| planta | endosperm | | | | | | | | | |
| Tissue | Dissected | | 37667 | 22101 | | 30004 | 31182 | | 3182 | 13348 |
| source | aleurone | | | | | | | | | |
| | Dissected | | 345983 | 166801 | | 566935 | 153805 | | 28742 | 57611 |
| | starchy | | | | | | | | | |
| | endosperm | | | | | | | | | |
| | Basal transfer | 11362 | | 24403 | 3882 | | 8619 | 0 | | 403 |
| | cell layer | | | | | | | | | |

| | | | | | | |
|---|---|---|---|---|---|---|
| Stage (DIV) | 6 | 15 | 6 | 15 | 6 | 15 |
| In vitro endosperm | 205702 | 23473 | 8872 | 1522 | 2606 | 295 |

Aleurone cells of the triple marker line were labeled by the expression of the ZsYellow1 fluorescent protein under the control of the barley Ltp2 promoter. Blast searches identified zmLtp2 as a putative homologue of the barley Ltp 2 gene, which is 45% identical to the predicted barley protein. According well with the observed pattern of expression for ZsYellow1 fluorescence in aleurone cells, zmLtp2 transcripts were present at increasing amounts as the endosperm developed, and was not detectable in the dissected starchy endosperm sample (Table 1). In contrast to the aleurone specific expression pattern of ZsYellow1 in aleurone cells, we detected the zmLtp2 transcript in the 27 DAP basal endosperm sample (Table 1). In our interpretation, this is most likely due to contaminating aleurone cells in this sample, since aleurone cells are juxtaposed to transfer cells and therefore very difficult to avoid when manually isolating transfer cells from basal endosperm. Similar to in planta, the barley Ltp2 driven ZsYellow1 marker is detectable exclusively in the aleurone layer of in vitro endosperm. At the time of initiation of in vitro cultures at 6 DAP, ZsYellow1 fluorescence is absent. At 2 DIV, weak yellow fluorescence appears in the aleurone layer, growing stronger during the next two days, until, at 6 DIV, all of the aleurone cells on the surface fluoresce brightly. The timing of the appearance of fluorescence in the aleurone layer of in vitro endosperm corresponds closely to the onset of fluorescence in aleurone cells of in planta endosperm, although the intensity of fluorescence in in vitro endosperm appears stronger at earlier time points than in planta (data not shown). The steady state level of the aleurone specific zmLtp2 transcript was significantly higher in 6 DIV endosperm compared to 12 DAP in planta endosperm (Table 1). To determine if this tendency was representative of other aleurone preferred or specific transcripts as well, we compared the steady state level of the aleurone-specific globulin1 (Belanger et al. (1989), Plant Physiol. 91, 636-643), the aleurone-preferred NLTP_MAIZE (Tchang (1985) Biochem. Biophys. Res. Commun. 133, 75-81) and THZ2_MAIZE (GAMMA-ZEATHIONIN 2) (Castro (1996). Protein Pept. Lett. 3, 267-274) transcripts (Table 2). All three were present at an increased level in in vitro grown endosperm compared to in planta, and in the case of NLTP_MAIZE, the transcript level significantly exceeded the level measured in dissected in planta aleurone layers (Table 2, 15 DIV compared to 27 DAP).

We interpret these data to show that in vitro grown endosperm retain the temporal and spatial control of aleurone and starchy endosperm cell fate specification similar to in planta. Furthermore, both morphological observations and transcript profiling data show that the proportion of aleurone cells over starchy endosperm cells increases in in vitro grown endosperm towards late developmental stages. Finally, we conclude that the ZsYellow1 and the AmCFP1 fluorescence in the triple line represent accurate developmental markers for differentiated aleurone and starchy endosperm cells, respectively.

In the triple line, red fluorescence from the End1::DsRed2 transgene first appeared in transfer cells around 15 DAP. In contrast, DsRed2 fluorescence failed to develop in in vitro grown endosperm. Microscopy sections through the area in which transfer cells normally develop confirm the lack of transfer cells in in vitro grown endosperm. The surface layers of cells in this region had turned brown and lacked the morphologically distinct transfer cell wall morphology typical of in planta transfer cells. The absence of cells with transfer cell morphology occurred whether the basal endosperm grew in direct contact with the agar medium or not (data not shown). In spite of the fact that red fluorescence was absent at the base of in vitro grown endosperm, the MPSS analysis detected the END1 transcripts at high levels both at 6 and 15 DIV (Table 1). Although originally reported as a basal endosperm specific transcript in barley, three lines of evidence suggest that the END1 gene is also expressed in aleurone cells. First, End1 transcripts are present in dissected aleurone layer samples at levels that are higher than expected from the presence of contaminating transfer cells. Second, the level of End1 transcript increases ten-fold between 12 DAP and 27 DAP, which is in accordance with other aleurone marker transcripts (Table 1). Thirdly, a weak red fluorescence signal in aleurone cells of the triple line support the conclusion that the End1 gene is expressed in aleurone cells. To further support the conclusion that transfer cells do not develop in vitro, we investigated the pattern of expression of additional transfer cell markers, including the new transfer cell specific transcript LTP_{—}895 and the two new transfer cell preferred transcripts defensin-like 3 and HSP 18 kDa-like (data not shown). Expression of all three markers was detected in total in planta endosperm and to a much higher relative level in isolated basal transfer cell layer samples. In contrast, the LTP_{—}895 marker was not detectable at all in vitro grown endosperm, whereas defensin-like 3 and HSP 18 kDa-like were present at significantly lower levels than in in planta endosperm. Similar to End1, we infer that these transcripts originate in aleurone cells, rather than in transfer cells. We conclude from these experiments that transfer cells do not develop in in vitro grown endosperm, suggesting that transfer cells require signaling from maternal tissues to fully develop.

### Materials and Methods

Plant Genotypes and Growth Conditions

The triple line was produced as follows. Transgenic maize lines expressing endosperm cell-type molecular markers were created using Agrobacterium tumefaciens-mediated transformation of immature High-II embryos (Armstrong et al. (1991) Maize Gen Coop Newsletter 65:92-93) as described (Zhao et al. (2001). Molecular Breeding 8:323-333. Binary vectors were created containing the following promoters and molecular markers: hvLtp2 promoter (Kalla et al. (1994). Plant J. 6:849-860) linked to ZsYellow1 (BD Biosciences Clontech, Palo Alto, Calif., USA) (Matz et al. (1999) Nat. Biotechnol. 17:969-973), 27 kDa γ-zein promoter (Ueda (1991) Theor. Appl. Genet. 82:93-100), (Russell (1997) 6:157-168) linked to AmCyan1 (BD Biosciences Clontech, Palo Alto, Calif., USA), and End1 promoter (U.S. Pat. No. 6,903,205) linked to DsRed2 (BD Biosciences Clontech, Palo Alto, Calif., USA).

The defective kernel 1(dek1-mum1) and supernumary aleurone layer1 (sal1-2) mutants were originally isolated from Pioneer Hi-Bred International's Trait Utility System for Corn (TUSC) (Lid et al. (2002) PNAS 99:5460-5465). Hemizygous transgenic T0 events were self pollinated to obtain lines homozygous at the transgenic loci. Homozygous lines expressing the transgene(s) were self pollination to generate endosperm materials. The self pollinated F2 of a cross between heterozygous dek/+ plants and the “triple line” was created to introgress endosperm cell-type molecular markers. Plants used for isolation of endosperm were grown under typical greenhouse conditions (68-82° F., 16 hour light, 1800 PAR) using a commercial potting medium (Metro-Mix 700, The Scotts Company, Marysville, Ohio, USA) and fertilized as needed with a standard fertilizer mixture (20-10-20 (N-P-K)). Two to three days after the first silks appear pollinations were made to prepare material for initiating in vitro endosperm culture.

Initiation and Growth of In Vitro Endosperm Cultures

Typically, ears were freshly harvested at 6 days after pollination (6 DAP) and surface sterilized by incubation with 70% (v/v) ethanol for at least 5 minutes prior to dissection. A scalpel is first used to slice open the tip of the kernel longitudinally and then forceps are used to split and remove the maternal tissues (pericarp and nucellar tissue) at the tip of the kernel to expose the embryo sac. The embryo sac can then be carefully lifted out of the surrounding nucellar tissue with the fine tip the forceps and placed immediately on culture media containing 4.3 g/l MS salts (GIBCO 11117), 0.5% v/v MS vitamins stock solution, 5 mg/l Thiamine HCl, 400 mg/l Asparagine, 15 g/l Sucrose and 3 g/l Gelrite (solid media), pH 5.8. In vitro endosperm cultures were incubated in darkness at 30° C.

Material for MPSS Libraries

Tissue samples from whole endosperm at 12 and 18 days after pollination (DAP), dissected aleurone from endosperm at 19 and 27 DAP, dissected Basal Endosperm at 12 and 27 DAP, and starchy endosperm at 19 and 27 DAP were collected, RNA prepared and mRNA isolated. Dissection of the aleurone, basal endosperm and starchy endosperm was aided by tissue specific marker genes, either anthocyanin expression (27 DAP aleurone and starchy endosperm) or fluorescent protein expression (see preceding description of the triple line). Endosperm cultures samples grown on solid media for 6 days in vitro (DIV) and 15 DIV were collected and mRNA isolated. A corresponding set of in vitro endosperms was collected from cultures grown on liquid media. Each of these mRNA samples was subjected to Massively Parallel Signature Sequencing (MPSS) expression profiling.

Normalization of MPSS Data

Counts reported for each 17 base pair signature sequence detected in a sample were divided by a normalization term and multiplied by 1 million to generate a tags per million (TPM) expression value. The normalization term is the total number of beads sequenced in the experiment minus the sum of the counts of the ten most common signatures. In the case of starchy endosperm cells that have a few genes with very high expression values, this normalization is superior to simply normalizing by the total number of beads because it mitigates the systematic reduction in counts of the non-seed storage protein genes and facilitates comparisons across tissue types.

Calculation of statistical significance values for MPSS data. A set of 6 samples that consisted of 3 pairs of biological replicates was used to construct a function relating the mean counts and the standard deviation of the mean. The curve derived from this function was similar to that reported by Stolovitzky and colleagues for human MPSS data (Stolovitzky et al. (2005) Proc. Natl. Acad. Sci. USA 102:1402-1407). Based on this function, t-values were computed for each signature sequence in a pairwise sample comparison. By comparing the t-value distribution for a pair wise comparison to that of a replicate sample comparison, False Discovery Rates (FDR) were computed for the each of the t-values.

Defining Endosperm Cell-Type Markers from MPSS Expression Data

Data from the six endosperm dissection samples (aleruone at 19 and 27 DAP, basal endosperm at 12 and 27 DAP and starchy endosperm at 19 and 27 DAP) were compared such that each sample was compared to the two different tissue samples with similar developmental stages (the 12 DAP sample was compared to the two 19 DAP samples). To derive a set of highly significant differentially expressed genes, we applied the criteria that a gene was considered preferential to a tissue (aleurone, basal endosperm or starchy endosperm) if the expression values of the gene in both samples enriched in that tissue were higher than in the corresponding samples from the other two tissues and the FDR of the four comparisons between the tissue samples with the highest expression and the two others was less than or equal to 0.1. These criteria produced a list of signatures that were further divided into two classes based on expression in the two non-preferred tissues. Specific genes were those with 0 counts in the other two tissues, and preferred genes were those with non-zero counts in either of the other tissues. This division is somewhat arbitrary in that the samples are not 100% pure and thus genes with very high levels of expression may be detected in the other two tissues due to a few cells of the other tissues remaining in the sample after dissection. The 17 bp signature sequences were subsequently mapped by exact sequence identity to a set of corn transcript sequences to produce the final gene list. From this list, three genes preferentially expressed in each of the three tissues were chosen based on detection in undissected samples and representation of multiple independent metabolic pathways or functions.

### EXAMPLE 7

### Temporal Control of Mitotic Divisions in the Periphery of In Vitro Grown Endosperm is Similar to that of In Planta Endosperm

To further document the similarity between in vitro grown and in planta endosperm we compared the progression of mitotic cell divisions in the periphery of the two types of endosperm. In planta, the frequency of mitotic cell divisions in the periphery of the endosperm peaks around 8 to 10 DAP (Mangelsdorf (1926) Bull. Conn. Agric. Exp. Stn. 279:513-614). First, we scored the mitotic index of dissected and fixed in planta endosperm, observing the expected peak at 8 DAP and reaching a very low level at 12 DAP. Mitotic activity of in vitro grown endosperm was recorded in cultures that were initiated from dissected endosperm harvested at 4, 6, 8 and 10 DAP. Observation of the frequency of mitotic divisions in this material was done by fixing samples with two days intervals. Independent of the developmental stage for initiation of the in vitro endosperm cultures, the mitotic activity appeared to follow the same pattern as observed in planta. For example, for endosperm placed in culture at 4 DAP, the mitotic index peaked after four days in culture, corresponding well with the in planta data. Also similar to in planta, the mitotic index reached very low levels after a total of 12 days of growth after fertilization. From these experiments we conclude that the in vitro grown endosperm is actively dividing under the culture conditions used, and that the temporal control of aleurone cell mitosis during the cell division phase is similar to that of the in planta endosperm.

Materials and Methods. The in vitro endosperm mitotic index measurements that are presented above were determined as follows. For each time point examined three endosperms from each of three ears were assayed. In planta endosperms were carefully dissected at 4, 6, 8, 10, and 12 DAP. In vitro endosperm cultures were initiated 4, 6, 8, 10, and 12 DAP and grown in culture on solid media as described. Following harvesting, endosperms were immediately fixed with 50% glacial acetic acid (v/v in in ddH2O) for at least 2 hours at room temperature. Samples were stored up to 48 hours at room temperature in fixative or in 70% alcohol at 4° C. for subsequent analysis. Assays were conducted by squash preparation on slides using one drop of Aceto-orcein stain (10 mg/ml orcein dissolved in 55% boiling glacial acetic acid) and dried using a 40° C. incubator followed by destaining with 5% glacial acetic acid. Mitotic activity was evaluated by counting the number of cells undergoing active mitosis from a random view of 100 cells. Two views were randomly selected for examination. Views for each sample were then averaged.

### EXAMPLE 8

### The Surface Rule of Aleurone Cell Formation: Surface Position is Necessary and Adequate to Specify Aleurone Cell Fate

Previous research has led to the conclusion that maize aleurone cell fate specification in planta occurs via positional signaling, that aleurone cells formed on all surfaces of endosperm irrespective of endosperm shape, and that aleurone cell formation did not require physical contact with surrounding maternal tissues) (Becraft et al. (2000) Development 127:4039-4048; Olsen (2004) Maydica 49:37-40). Two observations of the Ltp2 driven ZsYellow1 marker in in vitro cultured endosperm confirm and expand on these conclusions. First, whereas a strong and uniform yellow fluorescence was present in the aleurone cell layer of in vitro grown endosperm, the second cell layer fluoresced much weaker except for an occasional cell displaying a strong fluorescence signal. Our interpretation of these results was that the weakly fluorescing internal cells represented inner daughter cells of periclinally dividing aleurone cells. Such cells, upon translocation to a non-surface position, lost their aleurone cell identity, and hence their ZsYellow1 fluorescence as they converted to starchy endosperm cells. The second observation supporting the role of surface position in aleurone cell formation was based on the occasional formation of voids in the interior of the in vitro grown endosperm. Frequently, cells surrounding such voids displayed ZsYellow1 fluorescence, suggesting that they had assumed an aleurone cell fate. At higher magnification, it could be seen that these cells fluoresced brightly with ZsYellow1. In addition to ZsYellow1 fluorescence, these cells also possessed aleurone cell morphology. A more dramatic display of conversion from starchy endosperm cell fate to aleurone cell fate occurred when the interior starchy endosperm cells of in vitro grown endosperm failed to develop. In these cases, the surface on the interior side, originally starchy endosperm, had become covered with aleurone cells. Frequently, development of spherical bodies of endosperm consisting of one layer of aleurone cells covering an inner mass of starchy endosperm cells were observed on the interior surfaces. These structures were identical to the spherical bodies of endosperm present in defective kernel mutant seeds reported previously (Olsen (2004) Maydica 49:37-40). From these experiments, we concluded that aleurone cell fate is defined by surface position only, and that aleurone cells convert to a starchy endosperm cell fate when no longer in a surface position regardless of their previous position in the endosperm. Importantly, the ability to sense and respond to positional cues is an intrinsic property of the endosperm that is independent of short range signal input from maternal tissues.

### EXAMPLE 9

### Late Mitotic Activity in the In Vitro Endosperm Leads to the Formation of “Mini-Endosperms”

The mitotic index of the in planta endosperm surface layer drops dramatically after 8 DAP 3, which is consistent with observations in the literature. At later developmental stages, a low frequency of mitotic divisions in the surface layers of the endosperm has been reported, mitotic divisions occurring as late as 42 DAP (Mangelsdorf (1926) Bull. Conn. Agric. Exp. Stn. 279:513-614). These observations are also supported by studies of somatic mutations in anthocyanin genes, giving rise to colored or colorless sectors that demonstrate mitotic activity beyond 12 DAP (Levy et al. (1989) Dev. Genet. 10:520-531; Levy et al. (1990) Science 248:1534-1537). In order to monitor cell division activity in the in vitro grown endosperm beyond 6 DIV, we used particle bombardment to introduce the Ltp2::ZsYellow1 construct in 3 DIV cultures (endosperm harvested at 6 DAP, cultured for 3 days). Monitoring of cell division activity was done by scoring the number of cells per ZsYellow1 positive sector at 5, 7, 21 and 24 DIV. The results show that when we used 200 ng DNA/shot in these bombardments, a high frequency of fluorescent spots appeared after one day. After two days, 60% of the cells remained undivided, 20% had two neighboring fluorescent cells, which either represented two independent transformation events or daughter cells resulting from a mitotic division. Based on the observed increase in the frequency of sectors with more than two fluorescing cells at later stages, these data supported the conclusion from the mitotic index study that the most rapid phase of mitotic divisions occurs between 2 and 4 days after the bombardments, i.e. between 6 and 8 DIV. According well with the previously reported data from in planta endosperm, cell divisions also occurs in in vitro grown endosperm between 8 and 21 DIV.

As the in vitro cultured endosperm grew beyond 8 DIV, the surface of the endosperm developed bulges that entirely covered the exterior surface at 15 DIV. The first sign of these bulges are observable as sectors on the surface of the endosperm with varying intensity of the Ltp2 driven ZsYellow1 fluorescence. Soon thereafter, distinct bulges develop from the sectors. Sections of 15 DIV endosperm show that individual bulges consist of a surface layer of ZsYellow1 fluorescing cells with aleurone cell morphology, covering an interior mass of AmCyan1 fluorescing starchy endosperm cells. We refer to these structures as “mini-endosperms”.

To better understand the origin and development of mini-endosperms, we introduced Ltp2::ZsYellow1 DNA into young, in vitro grown endosperms by particle bombardment. In these experiments, we observed a low number of fluorescent spots that often developed into large sectors, and occasionally into mini-endosperms. Frequently, when observed over time, these sectors did not develop with mitosis occurring at a constant rate. Rather, they appeared to result from a sudden burst of mitotic activity over a short period of time. From these experiments we conclude that the ability of the endosperm to self-organize itself into structures with a surface layer of aleurone cells and an interior mass of starchy endosperm cells is retained throughout the life span of the in vitro endosperm cultures. Furthermore, it appears that mini-endosperm formation results from localized mitotic activity in the surface layers of the endosperm.

Materials and Methods. In the experiments described above, the in vitro endosperm cultures transformed by particle bombardment as follows. Endosperm cultures (3 DIV) grown on solid media were targeted with the PDS-1000 Helium Gun from BioRad at one shot per sample (each sample comprising 6 endosperms) using 650 PSI rupture disks. Approximately 200 ng of DNA (hvLtp2 promoter operably linked to ZsYellow1) was delivered per shot. Three replicate plates were produced and the cell fluorescence patterns were followed by assay of 3 random views of 100 cells of each culture.

### EXAMPLE 10

### Mutant Endosperm phenotype is retained in In Vitro culture

To further investigate whether mutant endosperm phenotypes impacting aleurone/starchy endosperm development are also displayed under conditions of in vitro culture, we cultured homozygous dek1 endosperm that contained the triple construct in vitro. Previous studies have shown that homozygous dek1 mutant endosperm lacks aleurone cells, although a low frequency of mutant endosperms can be found that contains aleurone cells expressing the GUS marker under the control of the barley Ltp2 promoter (Lid et al. (2002) PNAS 99:5460-5465). In line with this observation, we find that most dek1 mutant endosperm grown in vitro lack aleurone cells, and only occasionally do we find aleurone cells that are ZsYellow1 positive. In addition to these typical in vitro dek1 mutant endosperms, we also find a low frequency of mutant endosperms that appear more highly developed. In spite of the fact that these endosperm appears to contain a surface layer of aleurone like cells, only a very low frequency of these cells are ZsYellow1 positive. Notably, this type of dek1 mutant endosperm also forms mini-endosperms on the surface, demonstrating that the presence of fully differentiated aleurone cells is not required for mini-endosperm formation. Although proliferation that potentially could lead to mini-endosperm formation occurs in the aleurone like cells of these dek1 mutant endosperm, it is difficult to identify the exact cellular origin of the larger mini-endosperms. Interestingly, some of the mini-endosperm have an organized ZsYellow1 negative cell layer on the surface, the rest of the mini-endosperm lacking this level of organization. Our interpretation is that the Ltp2 promoter is a “late” stage molecular marker for aleurone cell differentiation and in this case a mini-endosperm has an organized surface layer that fails to fully differentiate into an ZsYellow positive aleurone cell due to dek1. Mini-endosperm structures without an organized surface layer are suggestive that fully differentiated aleurone cells are not required for formation of these structures. Homozygous sal1-2 mutant endosperms have two layers of aleurone cells, a phenotype that is also faithfully reproduced in in vitro endosperm culture. These experiments confirm that the molecular mechanisms involved with the phenotypes of the dek1 and sal1 endosperm are fully recapitulated in vitro.

### EXAMPLE 11

### Maize Endosperm Suspension Cell Cultures Retain Some Degree of Endosperm Cell Identity, But Not Organ Identity

In order to compare the level of organization in the tissue culture system for maize endosperm previously reported in the literature, we initiated cultures from a Ltp2::ZsYellow1 line at 6 DAP using the medium described by Shannon et al. ((1973) Crop Sci. 15:491-493). The most notable difference between this medium and the medium used here by us for in vitro endosperm organ culture is that it contains only 3% sucrose. After 15 DIV, notably more proliferation had occurred on the basal phase of the endosperm on the low sucrose medium compared to 15% sucrose medium used here. Mini-endosperm formation on the surface was rarely observed. Overall, these cultures appeared more heterogeneous that cultures on high sucrose, some embryo sacs giving rice to dense structures that fluoresced relatively strongly, suggesting the presence of aleurone cells. In addition to fluorescing cells, these structures also contained a new non-fluorescing cell type that was not previously observed on the high sucrose medium. Transverse section of these structures demonstrate that the strict organization of aleurone cells on the surface is not followed in these cultures, and that the non fluorescing cells are similar to highly vacuolated, non-differentiated cells typically seen in rapidly growing callus tissue. The second type of callus in these cultures consists of cells that are only loosely connected, lacking visible markers typical of in planta endosperm and resembling typical undifferentiated callus cells normally found in highly proliferative plant tissue cultures. Importantly, in contrast to high sucrose medium, where growth stopped after approximately 40 DIV, these cultures continued to proliferate, and are capable of establishing endosperm suspension cultures as described previously by (Shannon et al (1973) Crop Sci. 15:491-493). In conclusion, the experiments detailed above show that the high sucrose, but not the low sucrose medium, is able to maintain endosperm cell fate specification and organ identity.

### EXAMPLE 12

### Transient Expression in Embryo Sacs and Endosperm

A polynucleotide of interest is expressed transiently in the cultured embryo sacs or endosperm of the present invention. Transient expression of the product of a polynucleotide of interest is accomplished by delivering 5′ capped polyadenylated RNA corresponding to the polynucleotide of interest or expression cassettes comprising polynucleotide of interest operably linked to a promoter that drives expression in a plant cell. All of these molecules can be delivered using a biolistic particle gun. For example 5′capped polyadenylated RNA can easily be made in vitro using Ambion's mMessage mMachine kit. Following the procedure outline above, RNA is co-delivered along with DNA containing an agronomically useful expression cassette, and a marker used for selection/screening such as Ubi::moPAT˜GFPm::pinII. The cells receiving the RNA can be validated as being transgenic clonal colonies because they will also express the PAT˜GFP fusion protein (and thus will display green fluorescence under appropriate illumination). Plants regenerated from these embryos are then screened for the presence of the introduced gene.

### EXAMPLE 13

### Transformation of Mini-Endosperm

In this procedure, maize embryo sacs or portions thereof (e.g., endosperm) are isolated at 6 DAP and then in vitro cultured for 2 days on proliferation medium as described herein, prior to transformation by particle bombardment. Such in vitro cultured embryo sacs or portions thereof are bombarded with constructs containing a selectable marker gene (e.g., BAR) operably linked to the ubiquitin promoter, a fluorescent marker (e.g., ZsYellow) operably linked to the ubiquitin promoter and a candidate gene whose effect on endosperm development is to be tested, wherein the candidate gene operably linked to, for example, to the gamma zein promoter, which is known to be preferentially expressed in the endosperm. After bombardment, the embryo sacs or portions thereof are allowed to recover on non-selective medium for a period of time, for example two days, after which time the embryo sacs or portions thereof are transferred to a selective medium. Bulges or mini-endosperms that grow and are fluorescing represent stably transformed sectors. These structures are used to evaluate the effect of the expression of candidate gene on endosperm development. Alternatively, the candidate gene can be operably linked to the gamma zein promoter of the production to antisense transcripts so as to reduce the expression of the gene product of any endogenous gene. Another approach for downregulation of an endogenous gene is to use RNAi method.

Using the above described approaches, the effects of any candidate gene on mini-endosperm development can be evaluated. For example, the candidate gene that is overexpressed consists of the cytoplasmic portion of the Dek1 gene operably linked to the gamma zein promoter. The expected phenotype of the mini-endosperm is that it will have two layers of aleurone cells. To downregulate the expression of the endogenous Dek1 gene, an RNAi approach, for example, is used and the Dek1 hairpin construct is operably linked to the gamma zein promoter. The expected phenotype of the mini-endosperms or bulges is that they lack aleurone cells. In another example, the candidate gene that is down regulated using RNAi is the sal1 (supernumerary aleurone layers 1) gene with the gamma zein promoter operably linked to the sal1 hairpin construct. The expected phenotype of the mini endosperms is double layers of aleurone cells.

### EXAMPLE 14

### Use of In Vitro Cultured Endosperm for Chemical Genetics

In vitro cultured endosperm produced by the methods of the present invention and that express the ZsYellow fluorescent protein in aleurone cells under the control of the Ltp2 promoter are plated in 96 well microtiter plates with one endosperm added per well in liquid proliferation medium. To each well, a candidate chemical to be evaluated is added. The effect of the chemical on aleurone cell development is evaluated, for example, as the loss of aleurone cell identity as detected as lack of yellow fluorescence, either on the whole surface of the in vitro cultured endosperm or in patches on the surface.

For example, a chemical genetics approach can be used to assess the function of the Dek1 gene product. The defective kernel 1 (dek1) gene is required for aleurone cell development in the endosperm of maize grains and this gene encodes a membrane protein of the calpain gene superfamily (Lid et al. (2002) Proc. Natl. Acad. Sci. USA. 99(8):5460-5465). Using mutant sector analysis, it has been shown that, if an aleurone cell looses the Dek1 gene function, the cell loses its aleurone cell identity and converts to a starchy endosperm cell fate (Becraft et al. (2000) Development 127:4039-4048). n this example, in vitro endosperm cultures are exposed to the calpain inhibitor calpastatin by the method described above. It is predicted that exposure to calpastatin leads to lack of fluorescence in spot on the surface due to loss of fluorescence due to conversion of aleurone cells to starchy endosperm cells.

It is recognized that the method described above is not limited to chemical genetics, but could also include all chemical perturbations including hormones, growth substances, inhibitors, and the like. See, Avila et al. (2003) Plant Physiol. 133:1673-1676; and Armstrong et al. (2004) Proc. Natl. Acad. Sci. USA. 101(41):14978-14983; both of which are herein incorporated in their entirety by reference.

### EXAMPLE 15

### Use of In Vitro Cultured Endosperm for Subcellular Localization of the Protein Encoded by Crinkly4

A construct consisting of the maize Crinkly4 gene fused to the GFP protein is bombarded into the surface of in vitro cultured endosperm from an embryo sac that was isolated at 6 DAP and then in vitro cultured for 3 days. Fluorescence is observed in single cells the following day. The subcellular localization is identified to be to plasma membranes/cell walls using confocal microscopy.

The article “a” and “an” are used herein to refer to one or more than one (i.e., to at least one) of the grammatical object of the article. By way of example, “an element” means one or more element.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claims.

That which is claimed:

1. A method for culturing cereal endosperm comprising the steps of:
(a) isolating from a cereal plant a fertilized embryo sac comprising endosperm and an embryo; and
(b) exposing said fertilized embryo sac or portion thereof to a proliferation medium for an extended period of time so as to promote the growth and development of the endosperm or the growth and development of both the endosperm and the embryo.

2. The method of claim 1, wherein said portion comprises endosperm.

3. The method of claim 1, wherein said endosperm differentiates while exposed to said proliferation medium.

4. The method of claim 1, wherein said endosperm differentiates so as to form an aleurone cell layer surrounding starchy endosperm cells.

5. The method of claim 1, wherein mini-endosperm forms on the surface of said endosperm.

6. The method of claim 5, wherein said mini-endosperm comprises an aleurone cell layer surrounding starchy endosperm cells.

7. The method of claim 1, further comprising the step of removing said embryo from said fertilized embryo sac after step (a).

8. The method of claim 7, wherein said embryo is discarded after removal.

9. The method of claim 1, wherein the embryo is removed after the fertilized embryo sac has been exposed to said proliferation medium.

10. The method of claim 7, wherein said embryo is removed from said embryo sac by dissection.

11. The method of claim 1, wherein said extended period of time is at least about 5, 10, 15, 20, 25, 30, 35, or 40 days.

12. The method of claim 1, wherein said endosperm continues to grow and develop while exposed to said proliferation medium.

13. The method of claim 1, wherein said development of the endosperm comprises a member selected from the group consisting:
(i) the development of an aleurone cell layer,
(ii) the development of starchy endosperm cell,
(iii) the development of an aleurone cell layer and the development of starchy endosperm cells interior to said aleurone cell layer; and
(iv) the development of at least one mini-endosperm.

14. The method of claim 1, wherein said fertilized embryo sac is isolated from an ovule of said cereal plant by dissection.

15. The method of claim 14, wherein forceps are employed to isolate said fertilized embryo sac from said ovule.

16. The method of claim 15, wherein forceps are employed to remove said embryo from said embryo sac.

17. The method of claim 1, wherein said fertilized embryo sac is isolated from an ovule of said cereal plant at 0, 1, 2, 3, 4, 5, 6, 7, 8, 9, 10, 11, 12, 13, 14, or 15 days after pollination.

18. The method of claim 1, wherein said cereal plant is selected from the group consisting of maize, sorghum, wheat, rice, barley, oats, millet, rye, and triticale.

19. A method for culturing maize endosperm comprising the steps of:
(a) isolating from a maize plant a fertilized embryo sac comprising endosperm and an embryo, and
(b) exposing said fertilized embryo sac or portion thereof to a proliferation medium for an extended period of time so as to promote the growth and development of the endosperm or the growth and development of both the endosperm and the embryo;
wherein said proliferation medium comprises about a 1× concentration of MS salts, about a 1× concentration of MS vitamins, about 0.1 mg/mL BAP, about 10 to above 20% (w/v) sucrose, about 0.5 mg/L thiamine, and about 0.4 g/L asparagine.

20. A method for culturing mini-endosperm comprising the steps of:
(a) isolating mini-endosperm from cereal endosperm; and
(b) exposing said mini-endosperm or portion thereof to a proliferation medium so as to promote the growth and development of said mini-endosperm.
